(19) United States
(12) Patent Application Publication
(10) Pub. No.: US 2019/0112578 A1
(43) Pub. Date: Apr. 18, 2019

(54) Derivation and Self-Renewal of Multipotent Cells and Uses Thereof

(71) Applicant: IsletOne AB, Huddinge (SE)

(72) Inventors: Oscar Simonson, Stockholm (SE); Karl-Henrik Grinnemo, Hägersten (SE)

(21) Appl. No.: 16/096,967

(22) PCT Filed: Apr. 26, 2016
(86) PCT No.: PCT/EP2016/059291

§ 371 Date: Oct. 26, 2018

(51) Int. Cl. C12N 5/077; C12N 5/0775; A61K 35/28

(52) U.S. Cl. CPC C12N 5/0657; C12N 5/0662; A61K 35/28; C12N 2501/415; C12N 2533/52; C12N 2506/1346; C12N 2500/02

(57) ABSTRACT

The present invention pertains inter alia to novel efficient methods for culturing mesenchymal stem (stromal) cells (MSCs), wherein the MSCs have maintained and/or enhanced multipotency and/or maintain and/or enhanced immunomodulatory potential. The invention also pertains to cells obtainable by such methods, kits and compositions for carrying out the methods in accordance with the invention, and also medical applications and treatments using said cells.

### TECHNICAL FIELD

The present invention pertains inter alia to novel efficient methods for deriving multipotent cells, for instance mesenchymal stem cells or cardiac progenitor cells which may be Isl1 positive, (i.e. methods for inducing a cell to enter a multipotent cell lineage), for maintaining multipotency of various stem cells of mesenchymal origin in culture, methods for differentiating e.g. cardiac progenitor cells to all cell lineages normally residing in the heart, cells obtainable by such methods, kits and compositions for carrying out the methods in accordance with the invention, and also medical applications and treatments using the cells of the present invention.

### BACKGROUND ART

The turnover of cardiomyocytes is low in the adult heart but can increase during ischemia through upregulation of Islet-1 positive (Isl1+) or C-Kit positive (C-Kit+) multipotent cardiac progenitor cells (Genead et al., PlosOne 7, e36804 (2012)). The upregulation of endogenous progenitor cells is not sufficient to replace the damaged musculature after substantial ischemic damage, resulting in heart failure. An attractive approach to prevent the development of heart failure would be to provide progenitor cells capable of repairing tissue damage in the heart. These progenitor cells should be capable of giving rise to cardiomyocytes, smooth muscle cells, nerve cells and endothelium. The mammalian heart is derived from cardiac progenitors of the first and second heart fields (identified by TBX5, NKX2.5 and Islet-1 (Isl1) respectively) as well as from the pro-epicardium (TBX18). Isl1+ cells have this capacity and form ⅔ of the developing heart including the sino-atrial node (SA), part of the atrial-ventricular node (AV), right atrium, right ventricle, proximal aorta, trunk of the pulmonary arteries and proximal parts of the coronary arteries (Lam et al., Pediatr. Cardiol. 30, 690 (2009)).

However, one considerable limitation in using these progenitor cells for regenerative medicine pertains to the difficulty of expanding well-characterized clonal progenitor cell populations, from either adult tissue or from fetal/embryonic stem cell tissues. Laugwitz and coworkers have earlier showed that it was possible to expand postnatal Isl1+ cells from transgenic mice if they were co-cultured with cardiac mesenchymal feeder cells (Laugwitz et al., Nature 433, 647 (2005)). Furthermore, the inventors have also previously taught (in PCT/SE2013/000112) improved and inventive methods for generating Isl1-positive cells as well as cardiomyocytes, based on using a combination of a certain type of laminin and a Wnt-activating agent. Laminins have been used extensively in the art to culture-expand stem cells but the use of laminins is still rather unsophisticated and highly arbitrary, with the term laminin conventionally being employed to denote laminin 111 (which was the first laminin to be isolated and which forms part of the 3D matrix Matrigel®).

Consequently, there is still a great need in the art to improve methods for culture-expanding mesenchymal stem cells (MSCs) and to culture, derive and differentiate multipotent cells that can either give rise to e.g. cardiac-like cells, e.g. cardiomyocytes, both in vitro and in vivo. Furthermore, maintaining multipotency and immuno-modulatory properties of various types of stem cells, for instance mesenchymal stem cells (MSCs), is a key aspect behind several regenerative medicine strategies, and the current art lacks efficient methodologies for achieving this.

### SUMMARY OF THE INVENTION

It is hence an object of the present invention to overcome the above-identified problems and satisfy the existing needs within the art, i.e. to provide for facile and efficient derivation of MSCs and highly potent progenitor cells using clearly defined and highly controllable methods. Furthermore, the present invention enables not only efficient derivation of multipotent progenitor cells but also provides for substantially indefinite self-renewal and proliferation (i.e. clonal expansion) of various types of multipotent cells (essentially any type of stem cell, e.g. mesenchymal stem or stromal cells (MSCs)) as well as subsequent cellular differentiation, e.g. differentiation of cardiac progenitors into beating cardiac tissue both in vitro and in vivo. The cells obtainable using the methods in accordance with the present invention are highly suitable for various clinical applications and exhibit strong homing ability to tissues and sites of interest, for instance cardiac tissue post infarct. Specifically, the present invention discloses novel methods for the derivation of mesenchymal stem/stromal cells (MSCs) into Isl1⁺PDGFR-α⁺NKX2.5⁺ (multipotent cardiac progenitor) cells, and further differentiation into cardiac tissue as well as for maintaining stem cells (such as MSCs) in a multipotent state, e.g. maintaining multipotency and immuno-modulatory properties of any type of MSCs. The cells obtained using the derivation and differentiation methods according to the present invention home strongly to infarcted (ischemic) regions of the heart, where they become elongated and arrange in parallel with the surrounding cardiomyocytes. The present invention also relates to cells that are covered at least partially by certain laminins which may further increase their clinical potency and efficacy, and various applications in medicine of both laminin-coated cells but also the cells per se in accordance with the present invention. The multipotent cells obtainable via the methods of the present invention have strong immuno-modulatory capacity and can for instance induce switching of T cells to regulatory T cells, a feature that has never been described before for this type of MSC-derived multipotent progenitor cells. Moreover, the cells may express human leukocyte antigen G (HLA-G), a known immuno-modulatory molecule, which is known in the art to inhibit i.a. natural killer cell (NK cell)-mediated immune response.

In summary, the present invention relates to methods for culture-expanding essentially all types of MSCs with maintained and/or enhanced multipotency and with maintained and/or enhanced immuno-modulatory potential, wherein the methods comprise culturing MSCs and/or cells from a mesenchymal fraction under hypoxic conditions in the presence of at least one laminin isoform comprising an α5 laminin chain and/or in the presence of at least one laminin isoform comprising an α4 laminin chain. Furthermore, the present invention also relates to a method for culture-expanding MSCs comprising culturing MSCs in the presence of at least one laminin comprising an α5 chain and at least one laminin comprising an α4 chain, optionally under hypoxic conditions and said methods may also be used to derive highly potent multipotent progenitor cells from MSCs. Said progenitor cells may be cardiac progenitors, but other types of multipotent cells can also be culture-expanded and derived from mesenchymal stromal tissues and cells.

As above-mentioned, although hypoxic conditions are important for certain applications excellent culturing results have also been obtained using a combination of α5 and α4 laminins and normoxia. Further in brief, the present invention pertains to MSCs and multipotent progenitor cells that are not only multipotent but that also have immuno-modulatory capacity, for instance via switching T cells to regulatory T cells or/and through inhibition of NK cells. Additionally, the invention also relates to spontaneously beating cardiomyocytes, i.e. cardiomyocytes that beat spontaneously with a frequency of around 40-100 beats per minute (bpm). The cardiomyocytes as per the present invention appear to be excellent pacemaker cells, beating at around 70-90 bpm. Furthermore, upon in vivo administration of cardiac progenitors as per the present invention the cells home to sites of injury and align with the ischemic tissue. Without wishing to be bound by any theory, the migratory capacity of the cells is thought to be a result of interaction between SDF-1 and CXCR4, resulting in homing to sites of injury and inflammation. After homing to ischemic tissue, the progenitors may also elongate in the tissue in question.

Additionally, the present invention also pertains to medical uses and applications of said cells, preclinical and clinical kits and compositions for practicing the embodiments of the invention, and various other non-limiting aspects.

### BRIEF DESCRIPTION OF THE DRAWINGS

FIG. 1. Derivation of multipotent cardiac progenitor Isl1+ cells from embryonic rat cardiac mesenchymal stem cells and from rat bone marrow. (a) In the initial adherent stem cell fraction, there was only a small number of Isl1+ cells (<1%). During the first week of culture in hypoxic conditions in a Wnt-medium on laminin 511, laminin 521, or a combination of the two, the mesenchymal stem cells switched into cells expressing Isl1. Laminin 511 or laminin 521, or a combination of the two, was also combined with laminins comprising an alpha 4 chain (primarily LN-421 and LN-411), which resulted in ever better expansion in culture and derivation into Isl1+ cells. Along with the Isl1+ cells there were more differentiated cells expressing cardiac α-actinin. During subsequent passages, the relative proportion of cells expressing cardiac α-actinin was reduced and after two weeks in culture (5 passages), 80±15% of the cells (on LN-521 in combination with hypoxia), 85±8% of the cells (on LN-521+LN-421), 89±8% of the cells (on LN-521+LN-421 in combination with either hypoxia or normoxia) expressed Isl1 and there was no differentiated cells left. (b) Within two weeks of culture, the initial fraction had expanded 18 times. (c) Western blot analysis confirmed the activation of the Wnt/β-catenin pathway in the cultured Isl1+ cells, demonstrating increased levels of both phosphorylated Lrp6 on Serine 1490 and active (dephosphorylated) β-catenin (ABC) compared to the initial adherent cell fraction. In panel a, bars represent 50 μm.

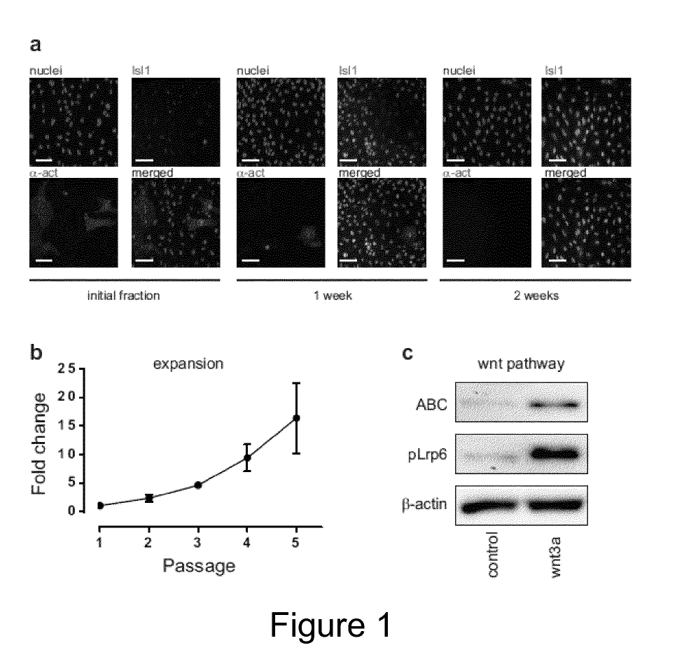

FIG. 2. Derivation of multipotent MSCs and progenitor cells from bone marrow, cord blood, and fetal and adult heart mesenchymal stem cells (MSCs). (A) Schematic representation of the experimental procedure used to generate and expand multipotent cells from an MSC source. (B) Before initiation of the Wnt and laminin (at least one as-containing laminin and at least one α4-containing laminin) protocol, less than 5% of the mesenchymal stem cell fraction expressed Isl1. In certain experiments, the cells may be mixed with cells expressing cardiac α-actinin (α-act), but that is an optional step. (C) During the first week in hypoxic culture on at least one α5-containing laminin, at least one α4-containing laminin, and in the Wnt-medium, the MSCs switched into cells expressing Isl1. (D) After two weeks of culture, on average 95±3% of the cells were Isl1 positive and more than 40% of the cells were still proliferating expressing Ki67. (E) The cultured cells could be differentiated into cells expressing cardiac α-actinin, von Willebrand factor (vWF) and smooth muscle actin (SMA). In order to verify the protocol, the initial mesenchymal stem cell fraction was cultured under different conditions: Wnt-medium+LN-521+LN-421+normoxia, Wnt-medium+LN-521+LN-411+hypoxia, Wnt-medium+LN-511+LN-411, Wnt-medium+plastic, medium without Wnt3a+LN-521+/−LN-511, and medium without Wnt3a+plastic. (G) The qRT-PCR-analysis demonstrates that the combination of Wnt-medium and at least one laminin comprising an α5 chain (e.g. either laminin 511 or laminin 521 or a combination of the two) is necessary for derivation and expansion of undifferentiated Isl1+ cells. The combination of at least one as-containing laminin and at least one α4-containing laminin resulted in enhanced derivation of Isl1 positive cells, and the addition of hypoxic conditions further improved the cell growth (data shown in FIGS. 8, 11, and 12). In panel B to E, nuclear staining with DAPI is shown in magenta. Cells costaining for Isl1 and magenta are shown in white. Bars represent 50 μm. In panel F, error bars represent mean±SD. In figure: ** p<0.01; *** p<0.001.

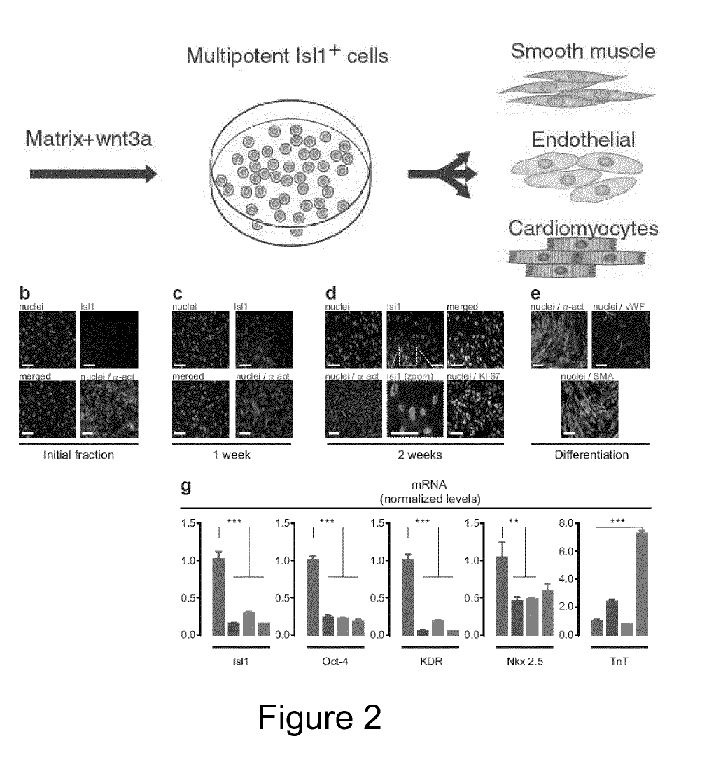

FIG. 3. Flow cytometry analysis of the adherent fraction from a human fetal/embryonic heart, adult cardiac tissue, and bone marrow aspirate from subjects. The cells expressed the mesenchymal stem cell markers CD105, CD90, CD73 and CD44, while being negative for the hematopoietic lineage markers CD14, CD19, CD34, CD45 and the endothelial marker CD31. Overall, the MSCs of the present invention in general adhered to the conventional MSC minimal criteria, namely plastic-adherent cells expressing CD105, CD73 and CD90, but not CD45, CD34, CD14, CD11b, CD79alpha, CD19 or HLA-DR.

FIG. 4. Heat maps representative of the gene-expression profiles of cultured multipotent progenitor cells in comparison to in vivo present cells in the Isl1-positive and -negative regions of a fetal heart. The origin of the cells is in (A) rat and (B) human. Average linkage and log 2 transformation of signals are presented. The heatmap color scale range from red (high expression) via black (average expression) to green (low expression).

FIG. 5. Heat map representative of the gene-expression profile and flow cytometry analysis defining different progenitor populations of cultured human multipotent progenitor Isl1+ cells. (A) Total RNA was isolated from Isl1-positive (Islet1+ve, red cells) and -negative (Islet-1−ve) regions from a human fetal heart (9.5 weeks) and through laser capture microdissection. In figure: OFT: outflow tract; LA: left atrium; LV: left ventricle: RA: right atrium; RV: right ventricle. Nuclei are stained blue by DAPI. Bar represent 50 μm. (B) Heat map: The cultured human multipotent cardiac progenitor Isl1+ cells (obtained from human fetal heart) had a higher expression of Isl1 than the Isl1-positive region of the human embryonic heart, and the expression of differentiation markers was much lower. The expression of pluripotency markers of the cultured Isl1+ cells increased during the culturing process (from 2 to 3 weeks). In figure, average linkage and log 2 transformation of signals are presented. The heat map color scale ranges from red (high expression) via yellow to blue (low expression). (C) In flow cytometry analysis of human fetal and adult heart, human cord blood MSCs, and human bone marrow MSCs, the CD34+ and CD45+ cell populations were excluded (which may be preferable under certain circumstances). The human Isl1+ cells co-expressed KDR, (around 60% SSEA-1) and around 5% C-kit. The SSEA-1+ cells co-expressed KDR, and they constituted around 3% of the C-kit+ cell population.

FIG. 6. Characterization of the cultured human multipotent cardiac progenitor Isl1+ cells using immunostaining and fluorescence-activated cell sorting (FACS). (A) Immunostaining demonstrates expression of Isl1, TBX18, and NKX2.5 in the cultured multipotent cardiac progenitor cells. (B) FACS analysis of the cells demonstrates subpopulations of cells expressing known markers stem cells KDR, c-kit, and SSEA-1. (C) Immunostaining confirms expression of SSEA-1 in some of the cultured multipotent cardiac progenitor Isl1+ cells; and shows expression of the known marker of cardiac multipotent progenitor cells PDGFRα, and CXCR4, a chemokine receptor that is involved in chemotactic activity of various cell types.

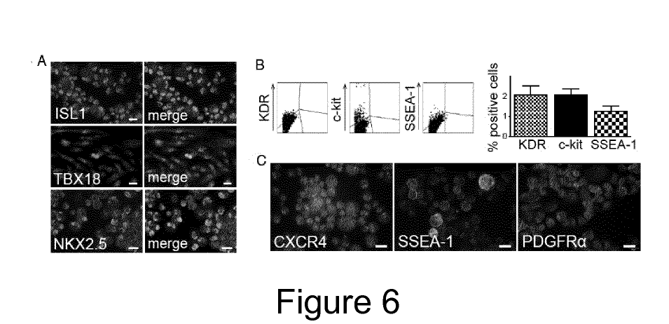

FIG. 7. Characterization of the cultured human multipotent MSC cells using fluorescence-activated cell sorting (FACS). Representative graph shows that interferon-γ (INF-γ) treated and untreated cells express HLA-G on the cell surface. A shift in HLA-G expression can be seen upon INF-γ stimulation, which demonstrate immunomodulatory responsiveness of the cells

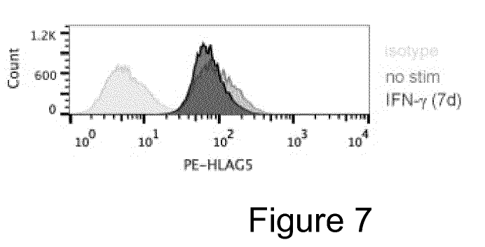

FIG. 8. Detection by immunohistochemistry of implanted rat multipotent MSC cells (obtained either from embryonic or fetal heart or from rat bone marrow) labelled with luciferase and β-gal expressing transposons. (A) Hematoxylin and eosin staining demonstrating the site of injection of labelled Isl1+ cells into the left ventricular wall. 24 hours after injection, X-gal staining identified the labelled multipotent cells (blue cells) both at the site of injection (insert in figure A) and in the outflow tract region close to the PA and Ao (insert in figure B). After two weeks, few multipotent cells could be detected at the site of injection (C). These cells had changed their appearance and were elongated and organized in parallel to the surrounding cardiomyocytes. (D, E) When labelled multipotent cells were injected into the peri-ischemic region of the left ventricle, the majority of the cells stayed in the infarction area (insert in figure D) and few cells were found in the outflow tract region 24 hours post injection (insert in figure E). (F) After two weeks, labelled cells were still found in the infarct area and again the implanted cells were elongated and interspersed between the surrounding cardiomyocytes (insert in figure F). In figures, bars represent 1000 μm and in insert 100 μm. Abbreviations; Ao: aorta; PA: pulmonary artery; RA: right atrium; LV: left ventricle.

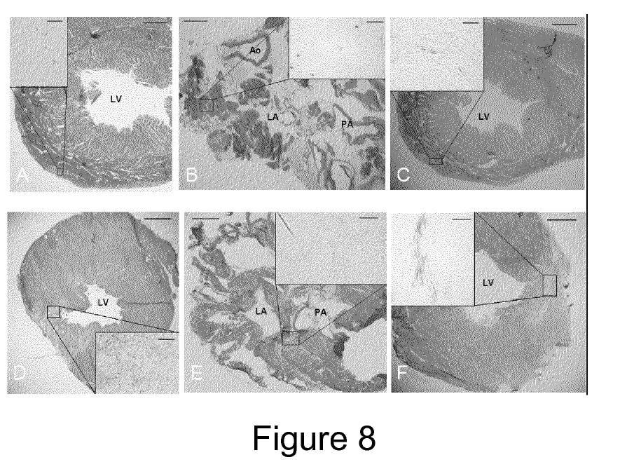

FIG. 9. Study of in vivo survival and migration of rat multipotent MSC progenitors (obtained either from mesenchymal fractions of bone marrow or from adult or embryonic heart) labelled with luciferase and β-gal expressing transposons. (A) Labelled cells were injected into the left ventricular wall of a normal rat heart, where the In Vitro Imaging System (IVIS system) detected a strong signal a few hours after injection. (B) After 24 hours, the strongest signal was detected in a region corresponding to the outflow tract, where it stayed during the detection period of one week (C). Labeled cells were in the next step injected into the left ventricular wall of a normal heart. Within 24 hours, the strongest signal was detected in the outflow tract region (D). After induction of myocardial infarction, the strongest signal was again detected in the ischemic region where it increased from 24 hours (E) to one-week post infarction (F). (G) Injection of labelled Isl1+cells into a tail vein, 8 hours after induction of myocardial infarction. The cells homed into the infarction region where the signal was strongest. Cells coated with a combination of various laminins (LN-521, LN-511, LN, 421, LN-411, LN-111, LN-211, and/or LN-221 and various combinations thereof, in particular LN-521 and/or LN-111/211/221 and optionally any one of LN-421 and LN-411) further improved homing, engraftment, and survival. Signals can also be seen in the lungs, which is probably indicative of some of the cells being maintained in the capillary network of the lungs.

FIG. 10. Laminin comprising an α5 chain is crucial for culture-expansion, self-renewal, and derivation of multipotent MSCs and hypoxic conditions drastically improves the cell growth, but the combination of at least one laminin comprising an α5 chain and at least one laminin comprising an α4 chain (optionally in combination with hypoxia) results in even more efficient culturing (culture-expansion) and derivation of MSCs. Laminin 511, laminin 521, and a combination of the two, induce MSCs (obtainable either from bone marrow of healthy human donors, from human cord blood, from the human amniotic sac, or from human fetal or adult heart tissue) to enter a multipotent lineage while retaining immuno-modulatory potential, whereas other laminins are incapable of promoting such cellular development. However, a combination of at least one laminin comprising an α5 chain and at least one laminin comprising an α4 chain (for instance LN-421 and/or LN-411) drastically further enhances culture-expansion and derivation of MSCs to multipotent immuno-modulatory cells that may be positive for Isl1 and/or HLA-G. Again, as can be seen from FIG. 8, applying hypoxic conditions may further improve the culture-expansion and derivation and retains the immuno-modulatory properties of the MSCs.

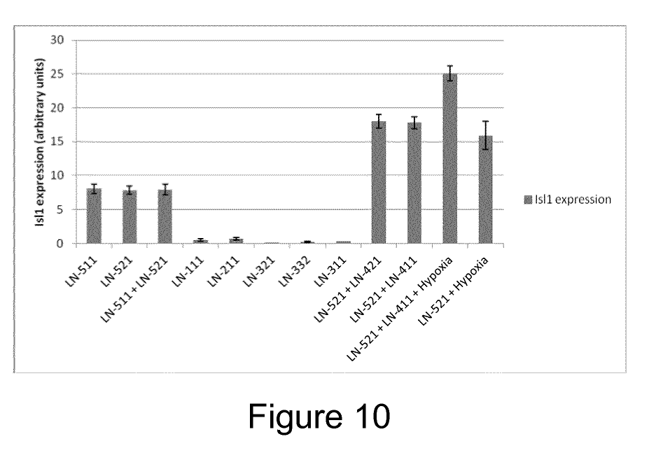

FIG. 11. Laminins 111, 211, 221, and a combination of the two promotes cellular differentiation including differentiation of multipotent cardiac progenitor cells. Laminin 111, laminin 211, laminin 221, or any combination thereof promote differentiation of multipotent cardiac progenitor (optionally Isl1+ cells) cells into cardiac cells (e.g. cardiomyocytes, smooth muscle cells, and/or endothelial cells). The differentiation protocol may also be combined with either normoxia or hypoxia.

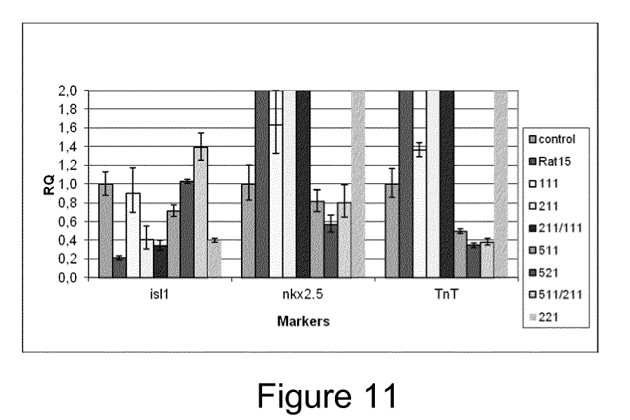

FIG. 12. Growth curves of multipotent cells (MSCs) (which may be Is1+ positive and/or positive for HLA-G) cultured under standard conditions (on plastic under normoxia) and on LN-521 under hypoxic conditions. Isl1+ cells in most instances proliferated significantly faster on LN-521 under hypoxia than under standard conditions.

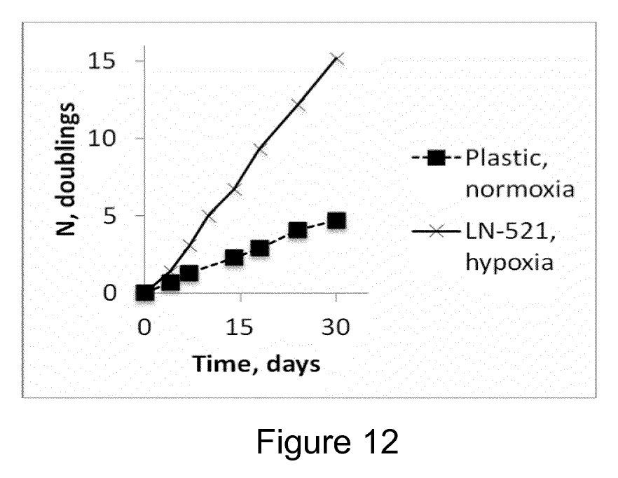

FIG. 13. Expression of markers of multipotency (Isl1, KDR, Nkx_2_5) and a marker of differentiated myocytes (Troponin T2) in cells grown under various conditions analyzed by qRT-PCR. The cells on LN-521 grown under hypoxia expressed significantly higher numbers of Isl1, KDR and Nkx_2_5 mRNA transcripts and lower numbers of Troponin T2 transcripts than the cells grown on LN-521 under normoxia, on plastic under hypoxia and on plastic under normoxia. Error bars represent 95% confidence interval.

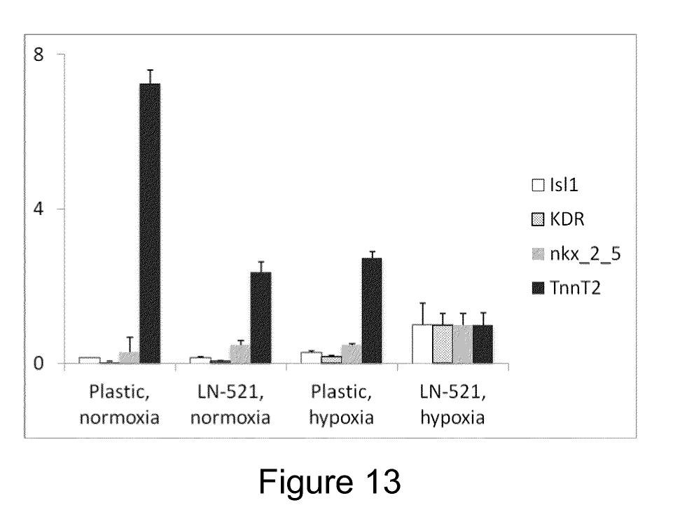

FIG. 14. Expression of markers of multipotency (Isl1, KDR, Nkx_2_5) and a marker of differentiated myocytes (Troponin T2) in Isl1 cells grown on alpha5-containing laminins and on mixtures of alpha5-containing and alpha4-containing laminins analyzed by qRT-PCR. Cells grown on the mixtures expressed higher levels of the multipotency markers and lower levels of the differentiation marker than the cells grown on alpha5-containing laminins only. Error bars represent 95% confidence interval.

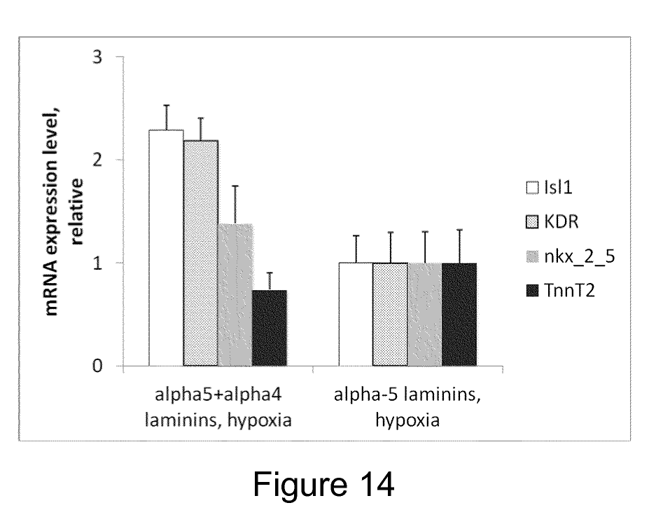

### DETAILED DESCRIPTION OF THE INVENTION

The present invention relates to methods for culturing MSCs (i.e. mesenchymal stromal cells or mesenchymal stem cells) and cells obtainable from a mesenchymal fraction with maintained and/or enhanced multipotency and/or with maintained and/or enhanced immuno-modulatory potential, comprising culturing MSCs under hypoxic or normoxic conditions in the presence of (i) at least one laminin comprising an α5 chain and/or (ii) at least one laminin comprising an α4 chain. Furthermore, the present invention also relates to a method for culture-expanding MSCs comprising culturing MSCs in the presence of at least one laminin comprising an α5 chain and/or at least one laminin comprising an α4 chain, optionally under hypoxic conditions. These methods may also be used to derive highly potent multipotent (progenitor) cells from MSCs, which is a key accomplishment in order to advance the field of regenerative cardiology.

Furthermore, the present invention pertains to multipotent MSCs that are not only multipotent but that also have strong immuno-modulatory capacity, for instance via switching T cells to regulatory T cells. Additionally, the invention also relates to immuno-modulatory cells (such as MSCs, multipotent cells such as progenitor cells (for instance cardiac progenitor cells), and/or differentiated cells such as cardiac cells) which are positive for HLA-G. Additionally, the invention also relates to cardiomyocytes that are beating, i.e. cardiac cells that beat spontaneously with a frequency of around 40-100 bpm. The cardiomyocytes as per the present invention are excellent pacemaker cells, beating at around 70-90 bpm under normal conditions. Furthermore, upon in vivo administration of cardiac progenitors as per the present invention the cells home to sites of injury and align with the ischemic tissue. Without wishing to be bound by any theory, the migratory capacity of the cells is thought to be a result of interaction between SDF-1 and CXCR4, resulting in homing to sites of injury and inflammation. After homing to ischemic tissue, the progenitors may also elongate in the tissue in question.

The present invention pertains inter alia to methods for deriving multipotent cells (e.g. methods for inducing a cell to enter a multipotent progenitor lineage), comprising the step of culturing a mesenchymal stem cell (MSC) (e.g. a cell from a mesenchymal cell fraction, obtainable for instance from bone marrow, Wharton's jelly, perinatal tissue, cord blood, amniotic tissue, adipose tissue, etc.) in the presence of at least one laminin comprising an α5 chain and/or at least one laminin comprising an α4 chain, under either hypoxic or in certain circumstances normoxic conditions. Optionally, the culture medium may comprise at least one agent, which activates the Wnt canonical pathway.

Further, the invention also relates to culturing of multipotent cardiac progenitor cells, which may be obtained using the methods of the present invention, in the presence of at least one laminin selected from the group comprising laminin 111, laminin 221, and laminin 211, or any combination thereof, in order to further differentiate the cell population, preferably to cardiomyocytes. Also, the present invention pertains to methods for self-renewal and/or proliferation of multipotent progenitor cells (optionally obtainable via the derivation methods as per the present invention but the self-renewal and/or maintenance procedure may also be applied to cells obtained via other methods or from other sources), comprising the steps of (i) culturing a multipotent such as an MSCs and/or a cell from a mesenchymal fraction (for instance any type of MSC which may be positive, for among other markers, HLA-G, or a cardiac progenitor Isl1⁺ cell of obtainable from a mesenchymal fraction) in the presence of at least one laminin comprising an α5 chain and at least one laminin comprising an α4 chain, and optionally in a medium comprising at least one agent which activates the Wnt canonical pathway, and (ii) maintaining and/or expanding the cells obtainable from step (i), in cell culture. The cell (which may be positive for inter alia HLA-G and/or Isl1+) obtained in steps (i) and/or (ii) (i.e. the self-renewal (proliferation) method) may subsequently be cultured in the presence of at least one laminin selected from the group comprising laminin 111, laminin 211, laminin 221, and a combination thereof, in order to further differentiate the cell population (into cardiac cells, specifically cardiomyocytes).

Furthermore, the present invention pertains to a method for culture-expanding any type of stem cell, including MSCs from any tissue, cells from a mesenchymal fraction, and/or multipotent progenitor cells such as cardiac progenitors, with maintained and/or enhanced multipotency, comprising culturing the MSCs under hypoxic conditions in the presence of at least one laminin comprising an α5 chain and/or at least one laminin comprising an α4 chain. Although hypoxic conditions are commonly utilized for culturing stem cells of various types hypoxia has never before been applied together with at least one laminin comprising an α5 chain and/or an α5 chain to maintain or increase multipotency of stem cells during culture. Culturing umbilical cord mesenchymal stromal cells under hypoxia and on Matrigel (which contains among other things laminin-111, a completely different molecule) has been described earlier by Chon and co-workers as a way of differentiating MSCs (Stem Cell Res Ther, 2013), i.e. making them less multipotent, which points in the complete opposite direction of the teachings of the present invention. The inventors of the present invention has made a serendipitous discovery in this regard, as the effects when combining at least one laminin comprising an α5 chain and hypoxic conditions are remarkable in how they unexpectedly maintain and/or increase multipotency of stem cells, and specifically mesenchymal stem cells (MSCs).

Furthermore, the present invention relates to a method for clonal growth and expansion of any type of stem cell, including MSCs and various types of progenitor cells, with maintained and/or enhanced multipotency, comprising culturing the stem cell under normoxic or hypoxic conditions in the presence of at least one laminin comprising an α5 chain. Clonal growth implies survival and expansion of an individualized cell, which may or may not be genetically manipulated.

Furthermore, the present invention also pertains to cells obtainable by the methods of the instant invention (e.g. MSCs, cells from a mesenchymal fraction, multipotent cells, progenitor cells which may be cardiac progenitor cells, Isl1+ cells and/or cardiac cells, for instance cardiomyocytes) for use in medicine, for instance in cardiology-related indications, as well as pharmaceutical compositions comprising the cells and at least one pharmaceutically acceptable excipient, in order to enable efficient delivery of the cells to the target site/tissue. Additionally, the present invention also relates to cells covered (at least partially) with at least one exogenous laminin, i.e. a laminin that is derived not from the cell or the cell culture but that is of exogenous origin and that is put in contact with the cells to enhance their in vitro and in vivo potency. The at least one laminin covering the cells may be selected from any one of LN-521, LN-511, LN-421, LN-411, LN-423, LN-211, LN-221, LN-111, etc., and any combination thereof. The present invention moreover relates to the use of a combination of at least one laminin comprising an α5 chain and at least one laminin comprising an α4 chain, and at least one agent which activates the Wnt canonical pathway, for deriving a multipotent cell (e.g. for inducing a cell to enter a multipotent lineage and optionally being positive for HLA-G and/or Isl1). Similarly, the present invention also relates to the use of a combination of (i) laminin 111, laminin 211, laminin 221, or a combination thereof, and optionally (ii) at least one agent which activates the Wnt canonical pathway, for differentiating a multipotent cell (optionally a cell positive for Isl1) into a cardiac cell. The agent which activates the Wnt canonical pathway may be included in the differentiation step although its presence is not absolutely necessary.

Where features, embodiments, or aspects of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the at least one agent which activates the Wnt canonical pathway described in connection with the method for deriving multipotent cells is to be understood to be relevant also for the kit of parts (interchangeably termed the kit). Furthermore, certain embodiments described in connection with certain aspects, for instance bromoindirubin-3′-oxime (BIO) as described in relation to the aspect pertaining to the method for deriving the multipotent cells, may naturally also be relevant in connection with other aspects and/or embodiment, in the case of BIO for instance in the aspects/embodiments pertaining to the compositions or the kit of parts, in accordance with the present invention.

For convenience and clarity, certain terms employed herein collected below. Unless otherwise defined, all technical and scientific terms used herein have the same meaning as commonly understood by one of ordinary skill in the art to which this invention belongs.

It shall be understood that within the context of the present invention the term “culturing [ . . . ] in the presence of” may be interpreted as encompassing contacting the cell/cells with e.g. at least one laminin comprising an α5 chain and at least one laminin comprising an α4 chain and/or at least one agent which activates the Wnt canonical pathway. The time of culturing in the presence of for instance the at least one agent which activates the Wnt canonical pathway may naturally be of importance for the present invention, as is outlined in greater detail below. Normally, the laminins used within the context of the present invention are coated on the cell/tissue culture equipment, e.g. a cell culture dish or stent that may be implanted into the human or animal body for a therapeutic purpose, for instance to derive Isl1⁺ cells and subsequently to differentiate said cells to cardiomyocytes, using the method steps of the present invention.

Further in accordance with the present invention, the mesenchymal stem cells (often referred to as mesenchymal stromal cells or multipotent stromal cells) (MSCs) (e.g. cells from a mesenchymal fraction) may be fetal or adult cardiac mesenchymal cells, embryonic stem cells, cord blood MSCs, perinatal MSCs, bone marrow-derived MSCs, and/or amniotic stem cells, tooth bud MSCs, MSCs derived from adipose tissue or Wharton's jelly, or any combination of these sources of cells. Mesenchymal cells, also known as mesenchymal stem cells (MSCs) are multipotent stromal cells that can differentiate into a variety of cell types, including but not limited to osteoblasts, chondrocytes, and adipocytes. MSCs may be derived from, as above-mentioned, the bone marrow, mesoderm, umbilical cord blood, Wharton's jelly, adult muscle for instance heart muscle, developing tooth buds, and/or amniotic fluid. Cells from the “mesenchymal fraction” shall within the context of the present invention be understood to relate to cells that may be derived from any of the above sources, e.g. bone marrow, mesoderm, umbilical cord blood, Wharton's jelly, adult muscle, developing tooth buds, and/or amniotic fluid. Bone marrow aspirate is a suitable source for cells of a mesenchymal cell fraction (i.e. mesenchymal cells). Bone marrow aspirate may be obtained through entering the crista iliaca with an aspiration needle, followed by extracting a certain volume of cell-containing aspirate, which may subsequently be sorted, cultured or frozen for future use. Alternatively, cord blood, adult heart muscle, or amniotic cells may be extracted and isolated for the purposes of the present invention. Isolation of said cells may be carried out in connection with partus through conventional techniques. The MSCs (i.e. the cells from a mesenchymal fraction) that are employed for treating a patient may be of allogeneic origin, i.e. it is normally not necessary to use autologous cells from the patient in need of treatment although autologous MSCs may be advantageous in certain settings and for certain purposes. The cells as per the present invention may further be obtained from a subject with a disease or disorder, for example from a subject with an acquired or congenital cardiac or cardiovascular disorder, disease or dysfunction, for example a cardiac defect. The cell may be of mammalian origin, for instance human, and it may also be a genetically modified cell. Additionally, as above-mentioned, the MSCs may also be obtained from a completely unrelated, matched or unmatched donor, preferable a young healthy donor (preferably below 35 years of age), for instance through aspirating cells (e.g. around 50 ml) from the bone marrow.

The term “laminin” pertains to a family of heterotrimeric glycoproteins composed of α, β, and γ chains that exist, respectively, as five, four, and three genetically distinct types forming around 15 different combinations in human tissues. They are named according to the chain composition; for example, laminin-511 (LN-511) consists of α5, β1, and γ1 chains. Earlier, laminins were named according to the order of their discovery. Thus, laminin-111 was named laminin-1; laminin-511 was named laminin-10; laminin-521 was named laminin-11. Laminins are the main component of basement membranes and are in contact with various cells in vivo and different laminins show various spatio-temporal expression patterns in developing organisms as well tissue-specific location and functions. Thus, laminin-211 and laminin-221 are primarily present in basement membranes around muscle cells and motor neuron synapses, laminin-332 is specific for subepithelial basement membranes, and laminin-511 is practically ubiquitous. The first discovered laminin, laminin-111, which is also known as laminin-1 or just laminin, is restricted to the early embryo and is a very rear isoform in adult human tissues. Laminin-111 has been studied most extensively, because it is possible to isolate it from the mouse Engelbreth-Holm-Swarm (EHS) sarcoma, which is easy to induce. Other laminins are hard to isolate from tissues due to extensive cross-linking and they should preferably be expressed as recombinant proteins in mammalian cells. The cellular effects of laminins are largely mediated via ligand binding to cell membrane receptors such as integrins, dystroglycan, and Lutheran glycoprotein. They can induce direct outside-in signaling in the cells, which has been shown to alter transcription levels of genes and even influence chromatin remodeling of the gene promoters. Laminins are very different in their ligand specificity. Thus, laminins containing an α5 chain can avidly bind integrin α6β1, integrin α3β1 and Lutheran glycoprotein, while laminin-111 (or laminin) has highest affinity to integrin α7X2β1 and dystroglycan. Laminin β and γ chains can modulate laminin-integrin binding, but the cellular interaction is mediated primarily via binding of the a chains to the receptors. Therefore, α5-containing or α4-containing laminins are highly different from, e.g. α2-containing or α1-containing laminins. α1-containing laminins have been used extensively in cell culture for a long time but bears no functional or structural resemblance to the laminins used for culture-expansion and/or Isl1⁺ derivation in the context of the present invention (i.e. α5-containing and/or α4-containing laminins).

Thus, the term “laminin comprising an α5 chain” used herein refers to any laminin polypeptide comprising an a chain of type five (5), e.g. laminin-511, 521, 523, etc. Additional laminins comprising an α5 chain are naturally also within the scope of the present invention. Similarly, the term “laminin comprising an α4 chain” as used herein refers to any laminin polypeptide comprising an a chain of type four (4), e.g. laminin-411, 421, 423, etc. Additional laminins comprising an α4 chain are naturally also within the scope of the present invention. More generally, all polypeptides and/or nucleotides disclosed in the present application encompass polypeptide and/or nucleotide sequences that have at least 70% sequence identity to the polypeptide and/or nucleotide in question, preferably a sequence identity of at least 80%, and even more preferably a sequence identify of at least 90%.

The terms “positive for” and “negative for” in the context of the present invention (e.g. a cell which is “positive for” a polypeptide and/or polynucleotide in question) shall be understood in accordance with the meaning normally given to the term within the biological and medical sciences, in essence a cell that is positive for a certain polypeptide and/or polynucleotide expresses said polypeptide and/or polynucleotide. The polypeptide and/or polynucleotide in question may be identified via various means, for instance using fluorescence-activated cell sorting (FACS) and/or immunohistochemical techniques and/or proteomics techniques such as LC-MS and/or 2D-PAGE. The term “positive for” may in certain instances be understood to comprise cell populations where at least 50% of the cells express the polypeptide (or polynucleotide or any other marker) in question, but preferably at least 70% or even more preferably at least 90% of the population expresses the polypeptide in question. The term “negative for” may, in the same vein, naturally be understood to be the opposite of the term “positive for”, i.e. at least 50%—but preferably at least 70% or even more preferably at least 90%—of the cells of the population shall not express the polypeptide (or other suitable marker) in question. However, for certain markers (e.g. polypeptides) the cell population as such may have a very low overall expression (e.g. 3%) of the marker in question although certain cells show upregulation of the marker.

The terms “Isl1⁺ cell” or “multipotent cardiac progenitor Isl1⁺ cell” or “multipotent cardiac progenitor cell” or “multipotent cell” may when relevant refer to a cell that may under normal circumstances express either one of the following markers: Isl1, Sox2, SSEA1, Nanog, Tra-1, Brachyury T, CXCR4, HLA-G, PDGFRα, and preferably KDR, or any combination of thereof (specifically combinations including Isl1, HLA-G and PDGFRalpha). Furthermore, cells in accordance with the present invention may also be positive for Tbx6, C-Kit, GATA4, Mef2c, Nkx2.5, Tbx1, Tbx18, Hand2, FoxH1, Fgf8 and/or Fgf10. Cardiac progenitor cells should preferably not express mature cardiomyocyte markers like troponin T (TnT), troponin I (TnI), myosin heavy chain (MHC) and cardiac α-actinin, nor should they express the mature endothelial marker CD31.

Furthermore, MSCs and other multipotent cells as per the present invention may preferably express indoleamine 2,3-dioxygenase (IDO) upon stimulation with IFN-gamma and/or TNF-alpha, they may switch T cells to regulatory T cells upon co-culturing with T-cells, and they may home to sites of injury and/or inflammation. Furthermore, such cells as per the present invention may also express HLA-G, which may contribute to mediating their strong immuno-modulatory properties. The cells as per the present invention may further display the following order of polypeptide abundance (i.e. the following order of abundance in expression of polypeptides when analyzed using proteomics techniques such as LC-MS): vimentin>Annexin A1, and preferably vimentin>Annexin A1>CD44>insulin-like growth factor binding protein 7>fatty-acid binding protein 3. Additionally, a population of the extracellular vesicle population in (b) displays the following order of polypeptide abundance: serotransferrin>annexin A2. Preferably, a population of extracellular vesicles obtainable from the Isl1+ cells may display the following polypeptide abundance: serotransferrin>annexin A2>connective tissue growth factor, and said extracellular vesicles may be virtually negative for at least one of the following polypeptides: LIM domain only protein 7, LIM domain and actin-binding protein 1, coatomer protein complex, subunit beta 2 (Beta prime), isoform CRA_b, ribonuclease inhibitor, PDZ and LIM domain protein 5, reticulocalbin-1, early endosome antigen 1, septin-2, actin-related protein ⅔ complex subunit 2, septin 11. These characteristics of the Isl1+ cells have never been described before and are highly important in the clinical application of the cells in accordance with the present invention. The ability of the Isl1+ cells to modulate the immune system, for instance via expression of insulin-like growth factor binding protein 7 (IGFBP7), which induces a switch of T cells to Tregs, may be a key contributor to therapeutic efficacy. Similarly, the expression of IDO (upon inflammatory stimuli) and the high expression of e.g. vimentin and Annexin A1 (as well as the other above-mentioned polypeptides) are important characteristics of the Isl1+ cells of the present invention.

The term “cardiac cell” or “heart cell” or “cardiomyocyte” shall be understood as a cell that may under normal circumstances express markers such as: myosin heavy chains (MyH), cardiac α-actinin, Troponin T (TnT) and/or Troponin I (TnI) and the proteins are normally organized in contractile elements. The cardiomyocytes are beating by around 40-100 beats per minute (preferably around 50-80 bpm) and have a rapid contraction phase and slower relaxation phase.

The term “progenitor cells” or “progenitors” is to be understood to refer to cells that have a cellular phenotype that is more primitive (i.e. is at an earlier stage along a developmental pathway or progression than is a fully differentiated cell) relative to a cell which it can give rise to by differentiation. Progenitor cells also frequently have significant or very high proliferative potential, and they can give rise to multiple distinct differentiated cell types or to a single differentiated cell type, depending on the developmental pathway and on the surrounding environment in which the cells develop and differentiate. A cell can begin as progenitor cell and proceed toward a differentiated phenotype, but then revert and re-express the progenitor cell phenotype. Consequently, a progenitor cell can be derived from a non-stem cell.

The term “stem cell” is to be understood to refer to an undifferentiated cell, which is capable of significant proliferation and to give rise to more progenitor cells having the ability to generate a large number of cells of the mother type which may in turn give rise to differentiated, or differentiable daughter cells. The daughter cells themselves can be induced to proliferate and produce progeny that subsequently differentiate into one or more mature cell types, while at the same time retaining one or more cells with parental developmental potential. The phrase “stem cell” refers to a subset of progenitors that have the capacity or potential, under particular circumstances, to differentiate to a more specialized or differentiated phenotype, and which retains the capacity, under certain circumstances, to proliferate without substantially differentiating. The term “stem cell” may generally refer to a naturally occurring mother cell whose descendants (progeny) specialise, often in different directions, through differentiation, e.g. by acquiring completely individual characters, as occurs in progressive diversification of embryonic cells and/or tissues. Cellular differentiation is a complex process typically occurring through many cell divisions and a differentiated cell may be derived from a multipotent cell which is itself derived from a multipotent cell, etc. While each of these multipotent cells may be considered to be stem cells, the range of cell types each can give rise to may vary substantially. Some differentiated cells also have the capacity to give rise to cells of greater developmental potential and such capacity may be be induced artificially upon treatment with various factors, or may be natural. Stem cells are also in many instances “multipotent” because they can produce progeny of more than one distinct cell type, but this is not required for their “stem-ness.” More in detail, multipotency can be defined as an ability of cells to differentiate into one or several types of terminally differentiated types of cells and/or as an ability to self-renew. Self-renewal is the other typical part of the stem cell definition, and it is important for the purposes of this invention. Theoretically, self-renewal can occur by either of two major mechanisms, i.e. symmetrically or asymmetrically. Asymmetric division refers to one daughter retaining the stem state and the other daughter expressing some distinct other specific function and phenotype. Symmetric division refers to the process when some of the stem cells in a population divide symmetrically into two stems, thus maintaining some stem cells in the population as a whole, while other cells in the population give rise to differentiated progeny only. It is formally possible that cells that begin as stem cells proceed toward a differentiated phenotype, but then revert and re-express the stem cell phenotype, a process which is often referred to as e.g. “dedifferentiation” or “reprogramming” or “retrodifferentiation”. Multipotency often manifests itself by the expression of markers for multipotency, for instance Isl1, KDR, SSEA-1, Sca-1, NANOG, PDGFRα, and/or Sox-2.

The term “differentiation” in the present context is to be understood to mean the formation of cells expressing markers or characteristics known to be associated with cells that are more specialized and closer to becoming terminally differentiated cells, which are incapable of further division or differentiation. Progressive differentiation or progressive commitment refers to the pathway along which cells progress from a less committed cell, to a cell that is increasingly committed to a particular cell type, and eventually to a cell that is terminally differentiated. Cell which are more specialized (for instance have begun to progress along a path of progressive differentiation) but not yet terminally differentiated are referred to as partially differentiated. Differentiation is a development process whereby cells acquire a specialized phenotype (for instance acquire one or more characteristics, features, or functions distinct from other cell types). In certain cases, the differentiated phenotype may refer to a cell phenotype that is at the mature endpoint in some developmental pathway (a so called terminally differentiated cell). In many (but not all) tissues, the process of differentiation is connected with exit from the cell cycle and in these instances, the terminally differentiated cells lose or greatly restrict their capacity to proliferate. For the purposes of the present invention, the term “differentiation” or “differentiated” is to be understood to refer to cells that are more specialized in their fate and/or function than at a previous point in their development, consequently including both cells that are terminally differentiated and cells that (although not terminally differentiated) are more specialized than at a previous point in their development.

The terms “renewal” or “self-renewal” or “proliferation” are used interchangeably herein, and are to be understood to refer to the ability of stem cells to renew themselves by dividing into the same non-specialized cell type over long periods, for instance many months to years. Proliferation may in some cases refer to expansion of cells by the repeated division of a single cell into two identical daughter cells.

The term “embryonic” may within the context of the present invention be used to refer to fetal material (i.e. cells or tissues), i.e. cells/tissues obtainable from a developing foetus. By definition, an embryo enters the foetal stage (i.e. becomes a foetus) at the time of organogenesis (i.e. when organs start to form) and the tissues and cells used in certain examples in accordance with the present invention are obtained from foetal material, i.e. from gestational week 6-10. Normally, the definition in humans is that the embryonic period stretches from fertilization to the eight week after fertilization, whereas the period starting from the eight week and lasting till partus is defined as the fetal period. Essentially, within the context of the present invention, when pre-natal cells and/or tissues are utilized they are normally of a fetal character, but sometimes also of an embryonic character.

The term “enhanced and/or maintained multipotency” as used herein may be understood as de-differentiation of a cell to a more multipotent state, or as maintenance of a cell at the same multipotency state as prior to exposing the cell to the conditions in question. The term “lineages” as used herein is to be understood to pertain to a cell with a common ancestry or cells with a common developmental fate. By way of a non-limiting example, a cell that has entered an “islet 1+ lineage” is to be understood to refer to the cell as being an Isl1⁺ progenitor and expressing Isl1⁺, and which may differentiate along the Isl1+ progenitor lineage restricted pathways. Such pathways may be one or more developmental lineage pathways, for instance an endothelial lineage, a cardiac lineage or a smooth muscle lineage. For example, a cell that has entered the Isl1+ lineage is a cell which is has the capacity of differentiating into three major cell types in the heart (i.e. cardiac, smooth muscle, and endothelial cells).

A “marker” is to be understood to describe the characteristics/features and/or phenotype of a cell and markers can often be used for selection/identification of cells comprising characteristics of interests. Markers will vary with specific cells. Markers are characteristics, whether morphological, functional or biochemical (enzymatic) characteristics of the cell of a particular cell type, or molecules expressed by the cell type. Markers are preferably proteins and more preferably possess at least one epitope for antibodies or other binding molecules that may be available. However, a marker may be any molecule found in or on a cell including but not limited to proteins (peptides and polypeptides), polysaccharides, nucleic acids, lipids, and steroids. Some examples of morphological characteristics/features or traits include for instance shape, size, and nuclear-to-cytoplasmic ratio. Examples of functional characteristics or traits comprise for instance the capacity to migrate under certain conditions, the ability to adhere to certain substrates, and the capacity to differentiate along particular lineages.

The terms “subject” and “individual” and “patient” may be used interchangeably herein and are to be understood to refer to an animal, for instance a human being, from whom cells can be obtained and/or to whom treatment, including prophylaxis or preventative treatment (for instance using the cells as per the present invention) is provided. Advantageously, the subject of the treatments as described in the context of the present invention is a mammal, preferably a human, or other mammals, preferably domesticated or production mammals.

Cardiovascular diseases, conditions or disorders are to be understood to comprise medical conditions related to the cardiovascular (heart) or circulatory system (blood vessels). A response to myocardial injury follows a clearly defined path in which some cells die while others enter a state of hibernation (wherein they are not yet dead but are dysfunctional). This is followed by infiltration of inflammatory cells and deposition of collagen as part of a scarring process. This happens in parallel with in-growth of new blood vessels and a certain extent of continued cell death. The term cardiovascular diseases is intended to comprise all disorders and diseases characterized by insufficient, undesired or abnormal cardiac function, for instance congenital heart disease and any condition which leads to congestive heart failure in a subject, particularly a human subject, ischemic heart disease, hypertensive heart disease and pulmonary hypertensive heart disease, and valvular disease. Insufficient or abnormal cardiac function can an implication of disease, injury, trauma, and/or aging, comprising for instance diseases and/or disorders of the pericardium, heart valves (for instance stenosed valves, incompetent valves, rheumatic heart disease, aortic regurgitation, mitral valve prolapse), myocardium (myocardial infarction, coronary artery disease, heart failure, angina, ischemic heart disease) blood vessels (for instance arteriosclerosis or aneurysm) or veins (for instance varicose veins, haemorrhoids). The term “ischemia” is to be understood to refer to any localized tissue ischemia due to reduction of the inflow of blood, and the term “myocardial ischemia” is to be understood to comprise circulatory disturbances caused by coronary atherosclerosis and/or inadequate oxygen supply to the myocardium. An acute myocardial infarction may represent an irreversible ischemic insult to myocardial tissue. This insult may result in an occlusive (for instance thrombotic or embolic) event in the coronary circulation, producing a milieu in which the myocardial metabolic demands are exceeding the supply of oxygen to the myocardial tissue. Various other diseases are amenable to treatment with the various cell populations as per the present invention. Such disease may be selected from acute respiratory distress syndrome (ARDS), graft-vs.-host disease (GvHD), solid organ rejections and rejections of cell and/or tissue transplants, inflammatory bowel diseases such as Crohn's disease and ulcerative colitis, rheumatoid diseases such as arthritis, any type of inflammation-driven or immunologically induced disease such as multiple sclerosis, ALS, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, or essentially any type of organ failure such as kidney failure, liver failure, lung failure, heart failure, and/or any combination thereof.

The term “agent” refers to any chemical, entity or moiety, comprising for instance (without any limitation) synthetic and naturally-occurring non-proteinaceous and proteinaceous entities. An agent may be a nucleic acid, nucleic acid analogues, peptides, proteins, antibodies, aptamers, oligomers of amino acids, nucleic acids, or carbohydrates comprising for instance oligonucleotides, ribozymes, DNAzymes, glycoproteins, proteins, siRNAs, lipoproteins, aptamers, and any modifications and combinations thereof. Agents are to also be understood to possibly include small molecules having certain chemical moieties, comprising for instance chemical moieties such as unsubstituted or substituted alkyl, aromatic, or heterocyclyl moieties including macrolides, leptomycins, and related natural products or analogues thereof. As used herein, the term “Wnt activating agent” refers any agent that activates the Wnt canonical pathway, or inhibits or suppresses the activity of inhibitors of Wnt canonical pathway. The activation is preferably selective activation, which means that the Wnt3 pathway is activated to the substantial exclusion of the effects (i.e. activation of inhibition) on non-Wnt3 pathways.

The term “therapeutically effective amount” is to be understood to refer to an amount which results in an improvement, alleviation, or remediation of the disease, disorder, or symptoms of the disease or condition.

The terms “administering,” “introducing” and “transplanting” are used interchangeably for the purposes of the present invention, for instance in the context of the placement of progenitors (for example Isl1⁺ cells) and/or cardiomyocytes differentiated using the methods of the present invention into a subject. A suitable method or route is one which leads to at least partial localization of the cardiovascular stem cells at a desired site. The cells may be administered (delivered) by any appropriate route which results in delivery to a desired location/tissue/site in the subject where at least a portion of the cells or components of the cells remain viable (the period of viability of the cells after administration may be as short as a few hours to a few days, to as long as several years). The modes of administration suitable for the purposes of the present invention comprise for instance (without limitation) intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrase “pharmaceutically acceptable excipient” as used herein is to be understood to relate to a pharmaceutically acceptable material, composition or vehicle, for instance a solid or liquid filler, a diluent, an excipient, a carrier, a solvent or a encapsulating material, involved in suspending, maintaining the activity of or carrying or transporting the subject agents from one organ, or portion of the body, to another organ, or portion of the body. The term “derivative” or “variant” as used herein is to be understood to refer to for instance a peptide, chemical or nucleic acid that differs from the naturally occurring polypeptide or nucleic acid by at least one amino acid or nucleic acid, for instance deletions, additions, substitutions or side-chain modifications, but at the same time retains one or more specific functions and/or activities of the naturally occurring molecule. Amino acid substitutions may comprise alterations in which an amino acid is replaced with a different naturally occurring or a non-conventional amino acid residue, and these substitutions may be conservative or non-conservative. The present invention pertains inter alia to use of agents that activate the Wnt canonical pathway. Examples of Wnt activating agents may include for example polypeptides, peptides, nucleic acids nucleic acid analogues, phage, peptidomimetics, antibodies, small or large organic molecules, ribozymes or inorganic molecules or any combination of thereof (optionally naturally occurring).The agents that activate the Wnt canonical pathway may include for example antibodies (polyclonal or monoclonal), neutralizing antibodies, antigen-binding antibody fragments, peptides, proteins, peptidomimetics, aptamers, oligonucleotides, hormones, small molecules, nucleic acids, nucleic acid analogues, carbohydrates or variants thereof that function to inactivate or activate one or more, as the case may be, nucleic acid and/or protein participant in a Wnt pathway as described herein or as known in the art. It shall be understood that the above described exemplifying embodiments can be modified without departing from the scope of the invention, inter alia with respect to the described constituents, materials, and process parameters applied.

The present invention is based in part on the unexpected realization that using a judicious combination at least one laminin comprising an α5 chain and at least one laminin comprising an α4 chain is highly advantageous and results in surprisingly enhanced derivation of MSCs (which may be positive for Isl1). When using the culturing methods of the present invention, MSC populations expand in culture over extended time periods with maintained and/or enhanced multipotency and/or with maintained and/or enhanced immuno-modulatory potential, which has never before been described in the art. The at least one laminin comprising an α5 chain may be selected from the group comprising laminin 511, laminin 521, laminin 523, any combination of said laminins, any natural, recombinant, or synthetic protein, which has at least approximately 70% (preferably at least 80% and even more preferably at least 90%) sequence identity to the polypeptide sequence of laminin 511 or laminin 521, any natural, recombinant, or synthetic protein comprising the polypeptide sequence of the laminin α5 chain G-domain, any natural, recombinant, or synthetic protein comprising a polypeptide sequence, which has at least approximately 50% (preferably at least 70% and even more preferably at least 90%) sequence identity to the polypeptide sequence of the laminin α5 chain G-domain, and any cell culture growth substratum or cell culture medium additive comprising polypeptides from the polypeptide sequence of laminin 511 or laminin 521 or laminin 523 or any other laminin trimer comprising α5 chain. The at least one laminin comprising an α4 chain may be selected from the group comprising laminin 411, laminin 421, laminin 423, any combination of said laminins, any natural, recombinant, or synthetic protein, which has at least approximately 50% (preferably at least 70% and even more preferably at least 90%) sequence identity to the polypeptide sequence of laminin 411 or laminin 421 or laminin 423, any natural, recombinant, or synthetic protein comprising the polypeptide sequence of the laminin α4 chain G-domain(s), any natural, recombinant, or synthetic protein comprising a polypeptide sequence, which has at least approximately 50% (preferably at least 70% and even more preferably at least 90%) sequence identity to the polypeptide sequence of the laminin α4 chain G-domain(s), and any cell culture growth substratum or cell culture medium additive comprising polypeptides from the polypeptide sequence of laminin 411 or laminin 421 or laminin 423 or any other laminin trimer comprising the α4 chain.

Unexpectedly, the inventors have found that culturing MSCs in the presence of at least one laminin comprising an α5 chain under hypoxia is important for extremely efficient culture-expansion with maintained and/or increased multipotency, and hypoxia also positively influences derivation of multipotent cells (e.g. MSCs or cardiac progenitor cells positive for Isl1), especially when cultured in the presence of at least one laminin comprising an α5 chain and at least one laminin comprising an α4 chain. When culturing cells in an atmosphere containing 10% or less (preferably 5% or less) oxygen (in essence, hypoxic conditions) expression of multipotency markers and markers for immuno-modulatory potential, after two weeks in culture, was stably detected in more than 90% of the cell population. Culturing of MSCs under hypoxic conditions on the combination of at least one laminin having an α5 chain and at least one laminin having an α4 chain allowed for proliferation of cells for more than 5 weeks, and sometimes even more than 10-20 weeks, and in certain cases over 1 year. The present inventors have described earlier, in WO2014/011095, how the use of at least one laminin having an α5 chain in combination with an agent activating the Wnt canonical pathway is an efficient technique for deriving multipotent cardiac progenitor Isl1 positive cells from MSCs, but the efficacy of the culturing protocol of the present invention (which combines laminins with α5 and α4 chains) is even higher and the cell growth even faster than what has previously been described, due to the serendipitous combination of α5- and α4-containing laminins, optionally during hypoxic conditions. This is highly unexpected as hypoxia is conventionally used to maintain multipotency of stem cells but not to induce derivation into a certain lineage (in this case an immuno-modulatory MSC lineage), making this a highly serendipitous discovery.

In a preferred advantageous embodiment, the combination of α5-containing laminin(s) (e.g. LN-511, LN-521, LN-523, etc., and any combination(s) thereof) and α4-containing laminin(s) (e.g. LN-411, LN-421, LN-423, etc., and any combination(s) thereof) seems to be optimized when the α5-containing laminin(s) constitute up to around 25% (by weight) of the total laminins. However, it is possible to decrease the amount of the at least one laminin comprising an α5 chain quite substantially, but in order to optimize the balance between cell growth and Isl1 derivation a concentration ratio of around 25% α5-containing laminins and around 75% α4-containing laminins. However, good results have also been obtained with 40% vs 60%, with 30% vs 70%, and with 20% vs 80%.

Further in accordance with the present invention, the at least one agent which activates the Wnt canonical pathway may be selected from the group comprising Wnt-1, Wnt-3a, Wnt-8, Wnt-8b, and any combination thereof. Additional agents activating the Wnt canonical pathway are naturally also within the scope of the present invention, irrespective of whether the activation of the Wnt canonical pathway is effected via direct activation of the pathway in question (for instance mediated by Wnt-3a) or via indirect stimulation of the Wnt canonical pathway by blockage of the non-canonical pathway (for instance via blocking glycogen synthase kinase 3 (GSK3) which causes an upregulation of beta-catenin) (i.e. using BIO). Also, various types of agents may be utilized within the scope of the instant invention, as is outlined in more detail below.

The inventors have realized that selecting an appropriate concentration interval of the at least one agent which activates the Wnt canonical pathway is important for the present invention, suitably a concentration of approximately 10-250 ng/ml, but preferably a concentration of approximately 50-150 ng/ml, or even more preferably approximately around 100 ng/ml. Naturally, the concentration interval depends on the activity and the potency of the Wnt-activating agent employed in the specific case. When utilizing BIO, either in combination with e.g. a Wnt-activating polypeptide (e.g. Wnt-3a) or alone, a suitable concentration range may be approximately 0.1-5 mM, preferably approximately 0.5-3 mM, or even more preferably approximately 2.5 mM. Furthermore, epidermal growth factor (EGF), at a concentration ranging from 1 ng/ml to 100 ng/ml (preferably around 10 ng/ml) may be included in the culture medium, in order to enhance cell expansion. Additionally, when DMEM/F12 medium is used for the cell culturing methods, the medium may be supplemented with B27.

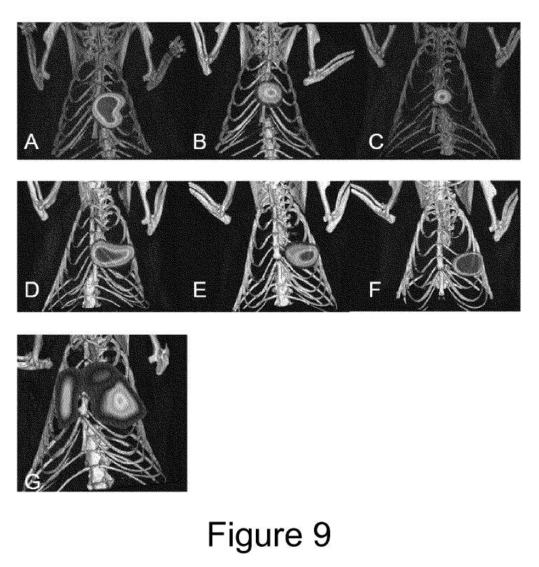

In order to further differentiate the cells obtainable by the methods of the present invention, a further differentiation step may be included, wherein multipotent cells (either obtained via the methods of the present invention or cells obtainable from any other source) is cultured in the presence of at least one laminin selected from the group comprising laminin 111, laminin 211, laminin 221, and any combination of thereof, and any natural, recombinant, or synthetic protein comprising a polypeptide sequence, which has at least approximately 70% sequence identity to the polypeptide sequence(s) of either laminin 111, laminin 221, or laminin 211. When culturing the cells in the presence of the at least one laminin selected from the group comprising laminin 111, laminin 211, laminin 221, and a combination thereof, and any natural, recombinant, or synthetic protein comprising a polypeptide sequence, which has at least approximately 70% sequence identity to the polypeptide sequence(s) of either laminin 111, laminin 221 or laminin 211, the Wnt-activating agent may be excluded from the culture medium, in order to further enhance differentiation to the desired cell type, in this case cardiac (heart) cells (and possibly tissues) (FIG. 9). Laminin 111 and other α1-containing laminins (such as laminin 121) (and matrices comprising such laminins, e.g. GelTrex®) may in some instances be utilized also to culture MSCs (optionally in combination with α4-containing laminin(s) (e.g. LN-411, LN-421, LN-423, etc., or any combination(s) thereof)) from mesenchymal tissues as per the present invention (in addition to their use for differentiating progenitor cells into cardiac cells), although derivation using a combination of α5-containing and α4-containing laminins is significantly more efficient and reliable.

In yet another aspect, the present invention pertains to cells obtainable by the methods of the instant invention, optionally further differentiated into e.g. cardiac cells, for instance cardiomyocytes (cardiac cells differentiated using the methods of the present invention normally display at least a 100-fold increase in Troponin T expression, and frequently a 150-fold increase in expression), in addition to optionally being positive for HLA-G. The cells as per the present invention may optionally be cryopreserved, in order to enable storage, facilitated handling, and subsequent use and experimentation.

In yet another aspect, the present invention relates to a composition and/or a kit comprising at least one laminin comprising an α5 chain and at least one laminin comprising an α4 chain, and optionally at least one agent which activates the Wnt canonical pathway. Further in accordance with the present invention, the at least one agent which activates the Wnt canonical pathway, and which is comprised in the composition, may be selected from the group comprising Wnt-1, Wnt-3a, Wnt-8, Wnt-8b, and any combination thereof. Additional agents activating the Wnt canonical pathway are naturally also within the scope of the present invention, irrespective of whether the activation of the Wnt canonical pathway is effectuated via direct activation of the pathway in question (for instance mediated by Wnt-3a) or via indirect stimulation of the Wnt canonical pathway. The composition may be a liquid, a solid, a matrix, a dispersion, a suspension, an emulsion, a microemulsion, a gel, or a solution, or any combination thereof. The composition in accordance with the present invention may for instance be a combination of a liquid (preferably comprising the at least one agent which activates the Wnt canonical pathway) and a substantially solid and/or a gel and/or a matrix (preferably comprising the at least one laminin comprising an α5 chain and the at least one laminin comprising an α4 chain).

In a further aspect, the instant invention relates to a kit (i.e. a kit of parts) comprising at least one laminin comprising an α5 chain and at least one laminin comprising an α4 chain, and optionally a cell culture medium optionally comprising at least one agent which activates the Wnt canonical pathway. The components of the kit (i.e. the laminins and the optional Wnt-activating agent) may advantageously be provided in separate containers (for instance vials, ampoules, tubes, or the like), in order to enable combining the components when carrying out the methods as per the present invention. The kit may for instance comprise a cell culture medium comprising the at least one agent which activates the Wnt canonical pathway (at a suitable concentration), and the at least one laminin comprising an α5 chain and the at least one laminin comprising an α4 chain coated e.g. on a surface suitable for cell culture (such as a cell culture plate, a well of a multi-well plate, a petri dish, a cell culture flask, etc.). Furthermore, the kit may also comprise additional components for differentiating the MSCs or multipotent cells, e.g. Isl1⁺ cells, to cardiac cells (e.g. cardiomyocytes), namely at least one laminin selected from the group comprising laminin 111, laminin 211, laminin 221, and any combination thereof. A suitable derivation and differentiation kit as per the present invention would thus comprise a cell culture medium comprising the at least one agent which activates the Wnt canonical pathway, at least one laminin comprising an α5 chain, at least one laminin comprising an α4 chain, a cell culture differentiation medium that may be devoid of the at least one agent which activates the Wnt canonical pathway, and at least one laminin selected from the group comprising laminin 111, laminin 211, laminin 221, and any combination thereof. In one embodiment, the laminins comprising an α5 chain and an α4 chain may be coated on a 1^{st} set of cell culture equipment, whereas the at least one laminin selected from the group comprising laminin 111, laminin 211, laminin 221, and any combination thereof, are coated on a 2^{nd} set of cell culture equipment, and where a cell culture medium comprising the at least one agent which activates the Wnt canonical pathway is provided in one container whereas cell culture differentiation medium devoid of the Wnt-activating agent(s) is provided in another container. However, judicious combinations of laminins comprising an α5 chain and laminins comprising an α4 chain and laminin 111, laminin 211, laminin 221 are also within the scope of the present invention.

Additionally, stents or other medical devices for implantation into a human or animal body may also be coated with suitable combinations of either the laminin comprising an α5 chain and at least one laminin comprising an α4 chain or the at least one laminin selected from the group comprising laminin 111, laminin 211, laminin 221, and any combination thereof, to enable derivation and/or cardiomyocyte differentiation inside the human or animal body. The stent may comprise for instance gold, cobalt-chromium, tanatalum, nitinol, silicone, polyethylene, and/or polyurethane or any combination thereof. The stents in question may e.g. be a coronary stent, a blood vessel bypass stent, and/or a trachea stent. The stent may be a drug-eluting stent, wherein the drug to be eluted may for instance be a Wnt-activating agent (for instance Wnt-1, Wnt-3a, Wnt-8, Wnt-8b, and any combination thereof), an agent for vascularisation, an immunomodulating agent, or any other suitable agent(s) to optimize the engraftment and stent function. The stents may be implanted with or without cells, e.g. MSCs, progenitor cells, cells positive for Isl1⁺ and/or PDGFRalpha and/or HLA-G or cells that have been further differentiated into cardiac cells (e.g. cardiomyocytes or cardiac endothelial or smooth muscle cells).

Furthermore, the multipotent cells and MSCs as per the present invention may be coated at least partially by exogenously derived laminins, in order to enhance their therapeutic effects. In one embodiment, MSCs may be covered in at least one laminin comprising an α5 chain, in at least one laminin comprising an α4 chain, in a combination of at least one laminin comprising an α5 chain and at least one laminin comprising an α5 chain, in any one of laminins 111, 211, 221, or any combination of all of the above laminins. The inventors have made the unexpected discovery that upon administration to a subject cells covered/coated by laminins exert stronger regenerative effects, which can also be seen when assessing the immuno-modulatory properties of the cells in vitro. The coating of the MSCs and/or the cardiomyocytes may be achieved by e.g. culturing the cells on temperature-regulated cell culture plates with so called UpCell™ surfaces (in essence a cell culture plate on which the cells become non-adherent upon a decrease in temperature (e.g. from 37 degrees C. to room temperature) or cultured in a matrix into which laminins are added before injection. These procedures result in attachment of the laminin to the cells, making them at least partially “coated” and/or “covered” in the at least one type of laminin. The coating or attachment of laminin to the cells is assessed by antibody-staining against laminins prior to administration to a subject. MSCs coated at least partially with α4-containing and/or α5-containing laminins and/or LN-111, LN-211, and/or LN-221 exhibit strong therapeutic efficacy, with improved homing to sites of injury and inflammation, enhanced engraftment, and overall a longer survival.

In another aspect, the present invention pertains to the cells as per the present invention for use in medicine. More specifically, the cells may be used in the treatment and/or prophylaxis of heart insufficiency, heart failure, myocardial infarction, and/or congenital heart disease due to cardiac defects affecting parts derived from the second heart field (for instance right atrium, right ventricle, outflow tracts (aorta, pulmonary arteries) and ventricular septum). Further, the instant invention also relates to pharmaceutical compositions comprising cells as per the invention, together with at least one pharmaceutically acceptable excipient, for use in medicine, for instance for use in the treatment and/or prophylaxis of heart insufficiency, heart failure, myocardial infarction, and/or congenital heart disease due to cardiac defects affecting parts derived from the second heart field (for instance right atrium, right ventricle, outflow tracts (aorta, pulmonary arteries) and ventricular septum), acute respiratory distress syndrome (ARDS), graft-vs.-host disease (GvHD), solid organ rejections and rejections of cell and/or tissue transplants, inflammatory bowel diseases such as Crohn's disease and ulcerative colitis, rheumatoid diseases such as arthritis, any type of inflammation-driven or immunologically induced disease such as multiple sclerosis, ALS, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, or essentially any type of organ failure such as kidney failure, liver failure, lung failure, heart failure, and/or any combination thereof Both the MSCs, obtainable through the derivation methods of the present invention (i.e. through culturing a mesenchymal cell in the presence of at least one laminin comprising an α5 chain and at least one laminin comprising an α4 chain, and optionally in a medium comprising at least one agent which activates the Wnt canonical pathway), and the cells that have been further differentiated using the differentiation methods of the present invention (i.e. through culturing in the presence of at least one of laminin 111, laminin 211, laminin 221, or any combination thereof) may be included in a pharmaceutical composition for the treatment or prophylaxis of various illnesses and ailments, not limited to the above-mentioned diseases and disorders. The pharmaceutical compositions as per the present invention may also further comprise at least one laminin, either as a free, solubilised agent or as a coating and/or attachment on the cells per se. Suitable laminins may be selected from the laminin(s) used in the culture methods of the present invention, for instance laminins comprising α5 or α4 chains (LN-521, LN-511, LN-411, LN-421) and/or any one of LN-111, LN-211, LN-221, or any combination thereof.

The pharmaceutical composition may comprise at least 100,000 (1*10⁵) cells per ml, preferably at least 500,000 (5*10⁵) per ml, in a pharmaceutically acceptable carrier, normally an aqueous solution comprising around 9% NaCl. Furthermore, preferably at least 100,000 cells may be used per kg of body weight, preferably at least 500,000 per kg of body weight, or even more preferably at least 1,000,000 MSCs per kg of body weight.

A further aspect in accordance with the present invention pertains to methods of treatment for improving, alleviating or preventing heart insufficiency, heart failure, myocardial infarction, and/or congenital heart disease due to cardiac defects affecting parts derived from the second heart field (for instance right atrium, right ventricle, outflow tracts (aorta, pulmonary arteries) and ventricular septum) in a subject in need thereof, the method comprising administering to the subject a therapeutically effective amount of the cells according to the instant invention. The subject is preferably a mammal, for instance a human being. The cells may be administered via intravascular administration, intravenous administration, intraventricular administration, epicardial administration, intramuscular administration, intraportal administration, intrathecal administration, and/or subcutaneous administration, or via any other suitable administration method that delivers the cells to the target site/tissue. Specifically, in the context of myocardial infarction, the cells may advantageously be administered to a patient via endovascular injection/infusion or via percutaneous administration in the occluded vessel (before or after re-opening), using a cathether. Additionally, for the treatment of sinus node dysfunction, Isl1⁺ cells (optionally differentiated into cardiomyocytes, in this case pacemaker cells) may be administered to a patient via percutaneous administration using a catheter introduced into the right atrium, preferably through the sinus coronarius.

Alternatively, multipotent cells of the present invention (which may for instance be MSCs and/or multipotent cardiac progenitor cells or cells further differentiated into cardiomyocytes) may be administered to a patient via the introduction of a suitable stent (e.g. a coronary stent that is introduced in connection with the treatment procedure). Furthermore, in utero administration of cells may be applied to treat fetal cardiac defects.

In a further aspect, the present invention pertains to the use of a combination of at least one laminin comprising an α5 chain and at least one laminin comprising an α4 chain, and optionally at least one agent which activates the Wnt canonical pathway, for deriving multipotent progenitor cells (i.e. for inducing a cell to enter a multipotent progenitor lineage). The multipotent cells, MSCs and the further differentiated cardiac cells generated and expanded by the methods of the present invention may be used in in vitro assays and experiments, for instance for screening agents, for example agents for the development of therapeutic interventions of diseases, including, but not limited to, therapeutics for congenital and adult heart failure. Alternatively, the cells of the present invention may be employed in assays for screening agents that are toxic to the cell (e.g. cardiotoxicity tests).

Experimental Section

In order to further improve feeder free culturing systems where the origin and expansion of multipotent cells could be followed, the inventors synthesized a plasmid construct comprising parts of the Isl1-promotor region linked to the fluorescent marker green fluorescent protein (GFP). The plasmid was introduced into the cells by means of electroporation. Mesenchymal cells (i) derived from rat embryonic hearts, (ii) obtained from rat bone marrow, (iii) obtained from the bone marrow of young healthy donors, (iv) obtained from human cord blood and other types of perinatal tissue, (v) isolated from human amniotic sources, (v) isolated from human adult cardiac tissue were labeled with the Isl1 gene construct concomitant to the addition of Wnt-containing medium (100 ng/ml Wnt-3a and 2.5 mM BIO). The cell culture plastic was pre-coated with various laminins comprising an α5 chain and laminins comprising an α4 chain (notably laminins 511, laminin 521, laminin 411, laminin 421, and any combination thereof). In culture the cells grew to confluency and were passaged every other day. At each passage, cells were analysed immunohistochemically for the expression of Isl1.

With each successive passage the ratio of multipotent cells increased, which also often corresponded to the Isl1 expression registered directly on the plate through activation of the Isl1 gene construct. In the initial fraction less than 1% of the cells expressed Isl1, which increased to more than 80% after two weeks in culture. Within two weeks, the multipotent cell population (which may be positive for Isl1) had expanded up to 25 times from the initiation of the Wnt+laminin-protocol and up to 50% of the cells were still proliferating expressing Ki67 at the end of the culturing process. The optimal ratio of laminins comprising an α5 chain and laminins comprising an α4 chain appears to be around 25% α5-containing LNs, to promote attachment and growth, and 75% α4-containing LNs for promoting MSC signaling.

To study the ability of the various MSC sources to give rise to multipotent cells, cells were labeled with the Isl1+ gene construct after the cells had grown to confluence and before being seeded on plates pre-coated with as-containing and α4-containing laminin(s) in accordance with the present invention. Additionally, 100 ng/ml Wnt-3a was again utilized to activate the Wnt canonical pathway, but this time BIO was excluded and the concentration of fetal bovine serum (FBS) was increased to 10%, cells proliferated in a similar manner to rat Isl1⁺ cells. These cells demonstrated a similar growth pattern as the rat cells and MSCs from other human tissues (i.e. mesenchymal cells obtainable from bone marrow, amniotic or cord blood cells), and after two weeks the cells had expanded almost 70 times from the initiation of the protocol, generating around 95% pure populations of multipotent cells expressing Isl1, NKX2.5, and PDGFRα.

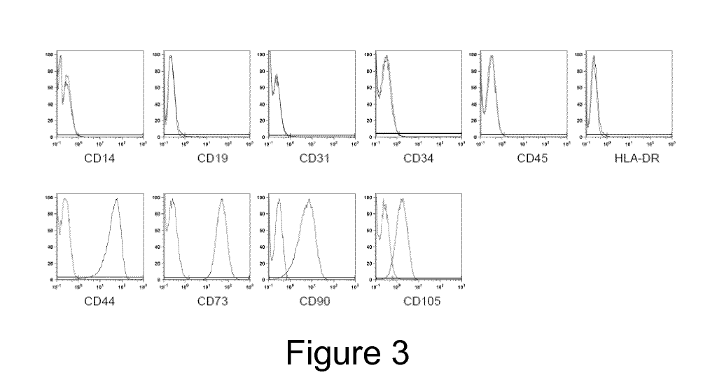

Adherent MSCs which were expanded for five weeks followed by FACS analysis using the mesenchymal panel. The adherent fraction expressed the mesenchymal stem cell markers CD105, CD90, CD73 and CD44, while being negative for the hematopoietic lineage markers CD14, CD19, CD34, CD45 and endothelial marker CD31 (FIG. 3). Similar expression profiles were obtained using MSCs from all the sources utilized. In order to ensure that the cells did not change their phenotype during expansion, samples of cells from the different sources were removed and stimulated according to the Wnt/laminin-protocol. This yielded similar amounts and ratio of multipotent cells expressing Isl1, NKX2.5, and PDGFRα. These findings lead us to conclude that multipotent stem cells and MSCs can be generated from various mesenchymal cell sources by stimulation of the Wnt canonical pathway when culturing on laminins, with optimal results when combining laminins comprising an α5 chain and laminins comprising an α4 chain and utilizing hypoxic culturing conditions.

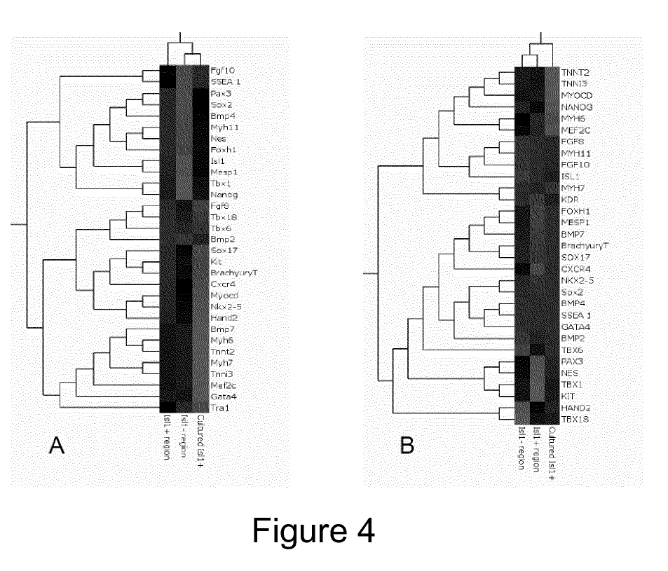

The gene-expression profile of cultured rat and human multipotent cells expressing Isl1, NKX2.5, and PDGFRα were studied and compared to the Isl1+ cells present in vivo in embryonic hearts (FIG. 4). These studies were performed in order to confirm that the cultured multipotent cardiac cells were similar to the Isl1+ cells that give rise to somatic cell types of the heart. In order to identify a core group of transcription factors that most probably are activated in Isl1+ cells, the inventors identified relevant transcription factors shown to be important for regulating the SHF development in mice. These studies have identified a set of transcription factors that are intimately involved in the transcriptional network of the SHF. These include members of the NK class of homeodomain proteins, the zinc-finger transcription factor GATA4, the MADS domain transcription factor Mef2c, T-box and Forkhead transcription factors as well as Hand class of basic-loop-helix (Hand) factors. Downstream to the Isl1 transcription factor, the Isl1-GATA4-Mef2c pathway seems to be playing a pivotal role for activation of the cascade that induces derivation of cardiomyocytes. The Isl1 and GATA4 factors seem to interact to induce activation of the Mef2c transcription factor, followed by upregulation of the transcription factors Nkx2.5 and Hand2. Other key-regulators of the SHF, are the Forkhead transcription factor FoxH1, the Tbx1 transcription factor, which is dependent on Forkhead factors for its activation and finally Fgf8, which in turn is regulated by Tbx1. Furthermore, blockage of Fgf-signaling prevents expansion of Isl1+ cells in the SHF, indicating an upstream effect on the Isl1-pathway. The cultured rat and human multipotent cardiac stem cells express the mesodermal markers TBX6 and Brachyury T, together with the above-mentioned key transcription factors involved in the transcriptional network of the SHF, i.e. Isl1, Mef2c, Nkx2.5, FoxH1, Tbx1, Fgf8 and Fgf10. Furthermore, they also express the early cardiomyocyte marker mesoderm posterior 1 (MESP1), while being negative for the more mature cardiomyocyte markers troponin I (TnI), troponin T (TnT) and myosin heavy chains (Myh) 6 and 7. The cultured multipotent cardiac stem cells obtained from the different sources employed herein (e.g. adult bone marrow, adult cardiac tissue, etc.), demonstrate a similar gene-expression profile as the Isl1+ cells isolated from the embryonic hearts. In the cultured human Isl1+ cells, the Isl1-GATA4-MEF2c pathway is more upregulated than in both the cultured rat multipotent cardiac stem cells and in the in vivo present Isl1+ cells. This correlates well with the upregulation of bone morphogenic peptides (BMPs), which are important for stimulation of early cardiogenesis and concomitant upregulation of the early cardiomyocyte markers Nkx2.5 and MESP1.

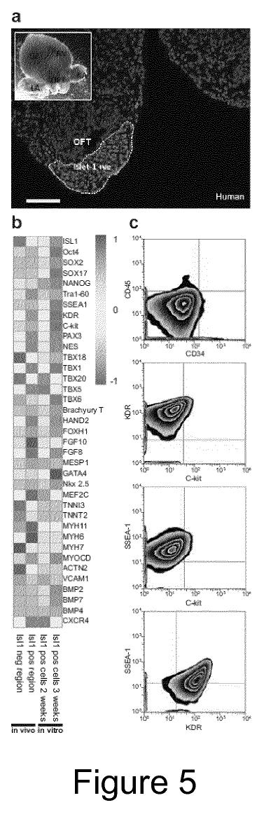

Other important findings are that both the cultured cells and the in vivo present Isl1+ cells express the pluripotency markers Tra-1, Sox2, Nanog and stage-specific embryonic antigen-1 (SSEA-1) while at the same time expressing nestin (NES), paired box 3 (PAX3), known to be expressed in neural crest cells and to a smaller extent the smooth muscle markers myocardin (MYOCD) and Myh11. According to micro-array data the cultured human and rat multipotent cardiac stem cells are multipotent stem cells with a genetic signature (Isl1+, KDR+, Nkx2.5+, Mef2c+, PDGFRα+) that has shown to be effective in differentiating into the three cardiovascular lineages with emphasis on cardiomyocytes. The cultured multipotent cardiac stem cells also express the stem cell markers C-Kit, the epicardial marker Tbx18, together with the multipotent surface marker SSEA1 and importantly the immunomodulatory polypeptide HLA-G. In prior studies the inventors have shown that Isl1+ cells are during the embryonic and post-natal period localized to the outflow tract (OFT). Even in the adult heart, resident Isl1+ cells can be found in this region and respond to ischemia by migrating from the OFT into the ischemic regions. This could imply that Isl1+ cells respond to myocardial ischemia by actively homing into this region. In order to follow the homing capacity of cultured multipotent cardiac stem cells in vivo and at the same time confirm this migration immunohistochemically, the cells were labelled with luciferase and β-gal expressing transposons by means of electroporation. Cells expressing luciferase can be followed in vivo with bioluminescent imaging and β-gal expressing cells can be stained for immunohistochemistry. By utilising the Sleeping-Beauty-100X (SB-100X) transposon system the transgenes were stably integrated into the genome. Labeled rat cultured multipotent cardiac stem Isl1+ cells, were injected into the left ventricle wall in immunoincompetent rats and the migration of the Isl1+cells was detected by an In Vivo Imaging System (IVIS® Spectrum CT) (PerkinElmer Inc., USA). Within 24 hours, the majority of the Isl1+ cells have started to migrate towards the outflow tract and to a lesser extent there were cells left at the site of injection (FIGS. 5A-C and 6A-C). In contrast, if the Isl1+ cells were injected into the peri-ischemic region induced by ligation of the left anterior descending artery (LAD), the majority of the cells did not migrate to the OFT. Instead they were found in the infarct where they at 2 weeks had become elongated and arranged in parallel to the surrounding cardiomyocytes. In order to further characterize the homing ability of Isl1+ cells, they were injected into the left ventricle of normal hearts and their migration followed with IVIS. Within 24 hours the Isl1+ cells were detected in the OFT, but after induction of myocardial infarction through LAD ligation, the cells migrated into the ischemic region. To further study the homing characteristics of Isl1+ cells, a myocardial infarction was induced followed by intravenous administration of labeled Isl1+cells through a tail vein. Again the cells migrated to the myocardial infarct. These results imply that the Isl1+ cells have the ability to home, especially to ischemic regions of the heart but also to areas where the Isl1+ cells are resident in the embryonic and adult heart. The explanation for this migratory response might be that during myocardial ischemia, the surrounding cardiomyocytes up-regulate CXCR4, which is the receptor for stromal cell derived factor-1 (SDF-1).

Mesenchymal stem cells are known to express SDF-1 and during ischemia these cells home from the circulation into the infarcted region stimulated through the SDF-1-CXCR4 axis. Moreover, engrafted mesenchymal stem cells secrete SDF-1, which were shown to cause homing of the cardiac progenitors into the infarct. According to our micro-array data, both cultured multipotent cardiac stem cells and especially in vivo present Isl1+ cells express CXCR4, which probably mediates the homing of the cells into the ischemic region. The SDF-1-CXCR4 axis might also be important for homing of Isl1+ cells into the OFT of a normal heart, since in vivo present Isl1+ cells have a high expression of this receptor. Furthermore, cultured multipotent cardiac stem cells were coated in vitro with various combinations of laminins (either α5-containing laminins, α4-containing laminins, α1-containing and/or α2-containing laminins, or various combinations thereof, notably LN-521+LN-421+/−LN-211) and these coated (covered) cells displayed even better homing and survival patterns.

In this study the inventors have shown that it is possible to derive pure populations of rat and human MSCs and also multipotent progenitor cells from mesenchymal cells from the bone marrow of healthy young donors, from mesenchymal cells obtained from cord blood, from amniotic sac sources, from Wharton's jelly, from adult cardiac tissue, and from embryonic/fetal cardiac mesenchymal cells in a culturing system where feeder cells have been replaced by laminins comprising an α5 chain (notably laminins 511, laminin 521, and a combination thereof) and laminins comprising an α4 chain (notably laminins 411, laminin 421, and a combination thereof), and wherein a culture medium comprising at least one agent that stimulates the Wnt canonical pathway is used. Additionally, the inventors also optionally applied hypoxic conditions to culture the MSCs and the multipotent stem cells in a highly efficacious manner, which is a completely novel approach. Both the human and rat multipotent cardiac stem cells have a similar gene-expression profile as their respective in vivo present counterparts, but they are more immature. The cultured multipotent cardiac stem cells have a genetic signature that is favorable for differentiation into the three cardiovascular lineages, they actively home into ischemic myocardium, displaying immuno-modulatory properties that are characterized by a certain proteomics profiles. In this study we have also devised a method for differentiating multipotent stem cells into cardiac cells (cardiomyocytes, smooth muscle cells, and endothelial cells), by including either laminin 111, 211, or 221, or any combination thereof, in the culture. These characteristics are beneficial for the use of multipotent stem cells to repair e.g. damaged hearts after myocardial infarction. Another field is the congenital heart diseases, where defects affecting the aortic arch, proximal pulmonary arteries and the outflow tract account for almost 30% of all congenital cardiac defects.

Animals and Ethics

The animal care committee of the Karolinska University Hospital approved all experimental animal procedures. Pregnant Sprague-Dawley (B&K Universal AB, Sollentuna, Sweden), gestational day 14 and Rowett nude rats (RNU, genotype rn/rnu) (Charles River Deutchland Inc., Germany) weighing 250-300 g were used in this study. To collect bone marrow tissue, heart tissue, and cord blood individual permission was obtained using a standard informed consent procedure and prior approval by the regional ethical committee. To collect human embryonic tissue (gestational weeks 5 to 10), individual permission was obtained using a standard informed consent procedure and prior approval by the regional ethical committee. The investigation conforms to the principles outlined in the Declaration of Helsinki.

Hypoxic Cell Culturing

Cells can be grown under hypoxic conditions using a variety of methods. Normally, hypoxic conditions for cell culturing are defined as culturing of cells in an atmosphere containing 5% or less of oxygen.

This was achieved in for instance the two following ways:
1) Culturing of the cells in an environment where hypoxia is achieved by injecting a chemically inert heavy gas, such as nitrogen, to reduce the oxygen concentration that the cells are exposed to.
2) Culturing of the cells in an environment where a premixed atmosphere is provided to fully replace the normal, oxygen-rich atmosphere.

Cloning of the Isl1-Promoter Construct and Transfection of the Mesenchymal Cells A 3 kb genomic human DNA fragment upstream of an Isl1 start codon (−3 to −3170 from ATG) was amplified by PCR with a high-fidelity Phusion DNA polymerase (Thermo Scientific). Forward primer 5″-aaa gag ctc GGT GTA ACA GCC ACA TTT -3″ and reverse primer 5″-gga gaa ttc CTG TAA GAG GGA GTA ATG TC-3″. The PCR product was cloned into the MCS of the vector pEGFP-1 (Clontech Laboratories Inc, USA) between the restriction enzyme cleavage sites SacI and EcoRI. The Isl1-plasmid was introduced into the rat and human cells using the Neon™ Electroporation system (Invitrogen, US). Pig pancreatic Isl1+ cells were used as positive controls, while Human Aortic Endothelial Cells (HAEC) were used as negative controls for the Isl1-gene construct.

Derivation and Expansion of Isl1+ MSCs from Embryonic Rat Hearts

For each experiment 10 pregnant sprague-dawley rats (gestational day, GD, 13 to 14) were used. The rats were euthanized in a CO₂ chamber after which the whole embryos (approximately 10 embryos/rat) were removed through a low midline abdominal incision. From each embryo the heart was removed under microscope, minced into small pieces and rinsed repetitively with Hank's buffered salt solution (HBSS) (GibcoBRL, NY, USA). The heart pieces were then predigested over night in 4° C. in 0.5 mg/ml Trypsin-solution in HBSS. The next step was to prepare the mesenchymal fraction, which is a modification of the protocol developed by Laugwitz and coworkers (Laugwitz et al., 2005). The predigested heart pieces were treated with collagenase type II (Worthington Biochemical Corp, Lakewood, N.J.) 240 U/ml in HBSS, 2-3 ml per round in the incubator at 37° C., for 10 to 15 minutes under gentle stirring. The supernatant was then centrifuged at 330-350 g for 8 minutes, resuspended in ice cooled HBSS. This procedure was repeated until the heart pieces were dissociated. The pooled cells were again washed, centrifuged at 330-350 g twice and resuspended in Dulbecco's Modified Eagle's Medium (DMEM) 4.5/M199 (4:1) (GibcoBRL) containing 10% horse serum (GibcoBRL) and 5% fetal bovine serum (FBS) (PAA Laboratories Inc., USA) with MycoZap (Lonza, Switzerland). The mesenchymal (adherent) fraction was separated from the cardiomyocytes and endothelial cells by two rounds of culture on plastic wells for 1 hour in incubator at 37° C., 5% CO₂ in 3% O₂. In order to follow the derivation of Isl1+ cells from the adherent fraction, these cells were labeled with the Isl1-gene construct containing the promotor for the Isl1-gene linked to the green fluorescent protein (GFP) gene (described above). This gene construct enables us to follow the derivation of Isl1+ cells directly in the culture dish. To stimulate the transformation of the mesenchymal cells into Isl1+ cells, the adherent fraction was detached using TrypLETMExpress (GibcoBRL) and recultured on plates coated with a thin layer of laminin 511, 521 or a combination of these, cultured in medium DMEM high glucose (GibcoBRL), MycoZap (Lonza), Hepes (25 mM) (GibcoBRL), glutamin (2 mM) (Fisher Scientific) and 15% FBS (PAA) in incubator at 37° C., 5% CO₂ in 3% O₂. At confluence (48 h), the cells were detached using TrypLETMExpress (GibcoBRL) and recultured on plates coated with Laminin 511, 521 or a combination of these, in Wnt-medium containing DMEM/F12 supplemented with 1 ml B27 (GibcoBRL), 2% FBS (PAA), MycoZap (Lonza), epidermal growth factor (EGF, 10 ng/ml) (R&Dsystems, Minneapolis, USA), 2.5 mM BIO (Sigma-Aldrich, USA), Wnt 3a (100 ng/ml) (R&Dsystems). At confluence, the cells were passaged and recultured on wells coated with laminin 511, 521 or a combination of these, in the same medium. At each passage, cells were harvested for analyses described below. After two weeks treatment with the Wnt medium, the culturing process was finished and cells harvested for further analyses or injections. Furthermore, we have also freezed and thawed adherent cells from different time points. Freezing of the cultured adherent cells was performed by detaching the cells and resuspending them after centrifugation in cooled Recovery™-cell culture freezing medium (GibcoBRL). The cells were transferred to storage vials and frozen gradually (−1° C. per minute) down to −70° C. and then transferred to liquid nitrogen (−180° C.). When the frozen cells were recultured, they were quickly thawed to 37° C., washed, and recultured on plastic plates coated with Laminin 511, 521 or a combination of these, in the same Wnt-medium and culturing conditions as described above.

Derivation and Expansion of Human MSCs and Multipotent Stem Cells

Aspiration of bone marrow cells was carried out as previously described from young healthy human donors. Cord blood cells and amniotic cells were collected and isolated using standard conventional procedures. Adult heart biopsies were obtained using conventional procedures. Abortions were performed according to a technique previously described by Westgren et al. The following procedures followed the same steps as for culturing of rat Isl1+ cells except that the pre-plating procedure was extended to 72 hours and performed in mesenchymal stem cell medium with DMEM low glucose (GibcoBRL), 10% FBS (PAA) and MycoZap (Lonza). As an alternative, culture medium free from non-human components may be employed, wherein the FBS is replaced by lyzed platelets obtainable from human blood. In order to follow the derivation of Isl1+ cells, the mesenchymal cells were labeled using the same Isl1-gene construct as for derivation of rat Isl1+ cells. To stimulate transformation into Isl1+ cells when relevant, we used similar culturing conditions as for rat Isl1+ cells with laminin 511, 521 or a combination, or any combination of laminin 511 or 521 and laminin 411 or 421, but with a Wnt-medium where BIO was excluded and serum level increased to 15% FBS (PAA) (or lyzed human platelets at a suitable concentration). After each passage cells were saved for further analyses and after 2 weeks the culturing process was finished and cells harvested for immunohistochemical staining and micro-array analyses. The mesenchymal cells from different time periods were also freezed and thawed as described for the rat Isl1+ cells.

Immunohistochemical Analyses of the Cultured Cells

At each passage and at the end of the culturing process, cells were cytospinned and analyzed immunohistochemically for the presence of Isl1+ cells and also Ki67 to detect cell proliferation. The cytospinned cells were initially stored frozen, followed by fixation in 4% formaldehyde, blocked with serum, and incubated with the primary antibodies; Isl1: goat anti-human Isl1 (R&D systems); Ki67: mouse anti-rat Ki67 (clone MIB-5, DakoCytomation, Denmark) and direct-conjugated mouse anti-human Ki67-FITC (clone MIB-1, DakoCytomation) respectively. To visualize the unconjugated primary antibodies, the sections were washed and incubated with the fluorescence-labelled secondary antibodies; Isl1: Alexa Fluor 488 rabbit anti-goat (Molecular Probes, Invitrogen) and for Ki67: Alexa fluor 568 goat anti-mouse (Molecular Probes). Staining with control antibodies, as well as staining of negative and positive control tissues, was done to verify the specificity of all antibodies.

Expansion of the Human Multipotent Mesenchymal Cells

The human mesenchymal stem cells (MSCs) from different sources were prepared according to the same protocol as described above. Instead of changing to the Wnt-medium, the MSCs were maintained in DMEM high glucose (GibcoBRL), MycoZap (Lonza), Hepes (25 mM) (GibcoBRL), glutamin (2 mM) (Fisher Scientific) and 15% FBS (PAA) for five weeks at 37° C., humidified air containing 5% CO₂ and 3% O₂. Cells were passaged twice during this time, followed by flow cytometry. In order to test that the correct MSC was expanded, cells were transferred to wells coated with laminin 511, 521, 421, 411 or a combination of these. The multipotency has been assessed by differentiation into the three mesenchymal lineages: bone, cartilage and adipose tissue. The immumodulatory properties has been assessed by expression of indoleamine 2,3-dioxygenase (IDO) and HLA-G, optionally in response to proinflammatory stimuli, e.g. INF-γ treatment.

Flow Cytometry

Approximately 0.85×10⁵ cells per single staining were washed in PBS, centrifuged once at 300 g for 5 min and incubated at 4° C. for 30 min with appropriate amounts of antibody. The labeled cells were then washed in PBS as above. For characterization of the mesenchymal fraction we used the following panel containing fluorochrome-conjugated monoclonal antibodies (mAbs) against the human surface antigens: CD31 (WM59), CD34 (581), CD73 (AD2), CD44 (G44-26), CD14 (MoP9), CD19 (HIB19), CD105 (266) (all from BD Biosciences, San Jose, Calif.); HLA DR (Tu36), CD45 (HI30) (Invitrogen) and CD90 (eBio5E10) (eBioscience Inc., USA). Each antibody-clone was titrated to optimal staining concentration using primary human samples. Data acquisition was done on a CyFlow ML (Partec GmbH, Munster, Germany), followed by data analyses with the FloJo software (TreeStar Inc., USA).

Laser Capture Microdissection (LCM)

Cryosections (7 μm) of whole rat embryos (GD 13), sliced in a transverse plane from head to tail and cryosections (7 μm) of a human embryonic heart (gestational week 9.5) and bone marrow-derived MSCs were collected onto sterile glass slides as well as on a 1-μm-thick PEN-Membrane-coated glass slides (Carl Zeiss, Germany). Targeted areas including both Isl1 positive and negative, were selected for capture using a 10× and 40× objectives. The negative regions were harvested from the developing left ventricle. Cryosectioning was performed using the 4-2-4-protocol, in which each slide was mounted with 3 sections. In using this protocol, two unstained membrane slides was preceded and followed by four cryosectioned slides, which were subsequently stained towards Isl1, using the same primary and secondary antibodies as for the cultured cells. These protocols aimed to histoanatomically localize the Isl1 positive and negative areas as well as generating high quality RNA yield. The micro-dissection process was performed through identifying the cells of interest, which in the next step were delineated and cut out through laser dissection.

Equivalent Isl1 positive and negative areas were laser captured and total RNA isolated using the PicoPure RNA isolation kit (PicoPure RNA isolation kit, Arcturus Engineering, Invitrogen) following the manufacturer's protocol. The quantity and quality of the RNA were determined using 2100 Agilent Bioanalyzer, RNA 6000 Pico LabChip (Agilent, Palo Alto, Calif., USA). All RNA samples were stored under sterile conditions at −80° C. for future analysis. All safety handling measures to avoid RNA degradation were fulfilled.

Microarray Analysis

Total RNA from cultured rat and human multipotent stem cells was isolated using the PicoPure RNA isolation kit (PicoPure RNA isolation kit) following the manufacturer's protocol as described in the previous section. Equivalent amounts (about 2 ng) of purified total RNA from micro-dissected tissues as well as from cultured cells were reversely transcribed and amplified using the Ovation Pico WTA system (NuGEN Technologies, CA, USA) following the manufacturer's instructions. Sense strand cDNA was generated using WT-Ovation Exon Module (NuGEN Technologies). cDNA was fragmented and labelled using the Encore Biotin Module (NuGEN). Labeled cDNA were hybridized to Affymetrix Rat and Human Gene ST 1.0 microarrays (Affymetrix Inc, CA, USA) respectively. GeneChips were washed, stained and scanned using the Fluidic Station 450 and GeneChip Scanner 3000 7G (Affymetrix). Preprocessing of the microarray data was performed in the Affymetrix Expression Console (v. 1.1) (Affymetrix Inc.) using the following methods: Summarization: PLIER; Background Correction: PM-GCBG; Normalization: Global Median.

Generation of Heatmaps

Heatmaps were created using Qlucore Omics Explorer 2.2. Hierarchical clustering of both samples and variables was done using the Euclidean metric and data where each variable was normalized to mean 0 and variance 1. Average linkage and log 2 transformation of signals were used. The heatmap color scale range from red (high expression) via black (average expression) to green (low expression).

Anaesthesia and Postoperative Care

RNU rats (genotype rn/rnu, Charles River Deutschland Inc.) used for intramyocardial injection of labeled rat Isl1+ cells were anesthetized with a subcutaneous injection of Midazolam (Dormicum, 5 mg/kg) (Algol Pharma AB, Germany), Medetomidin hydrochloride (Domitor vet, 0.1 mg/kg) (Orion Corp., Espoo, Finland), Fentanyl (0.3 mg/kg) (B.Braun Medical AG, Seesatz, Switzerland) and subsequently endotracheally intubated to be able to perform the intramyocardial injections and induce myocardial infarctions described below. Positive-pressure ventilation was kept at a rate of 100 cycles per minute with a tidal volume of 4-5 ml with room air using a ventilator (7025 Rodent ventilator, UGO BASILE S.R.L, Italy). The anesthesia was reversed by an intramuscular injection of Flumazenil (Lanexat, 0.1 mg/kg) (Hameln Pharma, Germany) and Tipamezol hydrochloride (Antisedan vet 5 mg/kg) (Orion Corp., Espoo, Finland). Postoperative analgesia was maintained by administrating Buprenorphin hydrochloride (Temgesic, 0.004 mg/kg/twice per day for 3 days) (Schering-Plough Corp., Belgium). Rats that showed signs of malfunction were excluded from the study.

Study of In Vivo Survival and Migration of Labeled Isl1+ Cells

The RNU rats were divided into four groups depending on how the Isl1+ cells were injected. In each experiment the hearts were exposed through a left thoracotomy. In the myocardial infarction groups, the left anterior descending artery (LAD) was permanently ligated and infarction induction was confirmed by color change and dyskinesia of the antero-lateral wall of the left ventricle. In each experiment 1 million labeled Isl1+ cells (obtained either from cardiac tissue or from the bone marrow) were used except for the intravenous experiment where 4 million cells were injected. Before injection, the Isl1+ cells were labelled with 2 μg pT2/C-fluc (Addgene, US), 2 μg pT2-β-gal and 1 μg SB 100× using the Neon™ Electroporation system (Invitrogen). In order to detect the cells after implantation into the myocardium, D-Luciferin (300 mg/kg) was injected intraperitoneally, followed by 15 min incubation. Bioluminescence imaging was in the next step performed in the IVIS® Spectrum CT (Perkin Elmer Inc.) using 5 min exposure and high sensitivity settings. The rats were imaged in a ventral position to be able to detect the expression of the transplanted cells and quantify the total flux using the Living Image Software (Perkin Elmer Inc.). The corresponding CT image was performed on a Quantum FX μCT (Caliper, Perkin Elmer Inc.) with 17 s scan time. The luminescent images were superimposed on the corresponding CT scan.

The rats were divided into the following groups: 1) injection into the left ventricular wall of a normal heart (n=4); 2) Injection into the peri-ischemic region of the left ventricle, directly after induction of a myocardial infarction (n=4). Cell survival and distribution were followed by IVIS (Perkin Elmer Inc.) and the hearts were harvested at 1 and 2 weeks for immunohistochemical analysis to confirm the IVIS data; 3) Injection into the left ventricular wall of a normal myocardium, followed three days later by induction of a myocardial infarction through a re-thoracotomy and LAD ligation (n=3); 4) Induction of a myocardial infarction followed by intravenous injection through the tail vein 8 hours after infarction induction (n=2). Since cell survival and distribution were found to correlate well with the IVIS signal, the animals in group 3 and 4 were only followed with IVIS for 1 week.

Detection of Rat Isl1+ Cells After Intramyocardial Injection

The hearts were harvested and freeze-sectioned into 7 μm thick sections. Hematoxylin and eosin stainings were used to get an overview of the different areas and subdomains of the hearts. From these sections, it was then possible to direct the X-gal staining to the regions of interest. The X-gal staining was done using a β-gal-staining kit (K1465-01) (Invitrogen) following the manufacturer's protocol.

1. A method for culturing mesenchymal stem cells (MSCs) and/or cells obtainable from a mesenchymal fraction, comprising culturing MSCs and/or cells from a mesenchymal fraction under hypoxic conditions in the presence of (i) at least one laminin comprising an α5 chain and/or (ii) at least one laminin comprising an α4 chain.

2. The method according to claim 1, wherein the at least one laminin comprising an α4 chain constitutes at least 50% (w/w) of the total laminins, preferably at least 70% of the total laminins.

3. The method according to any one of claims 1-2, wherein the methods are used for deriving multipotent progenitor cells, for instance cardiac progenitor cells.

4. The method according to claim 3, wherein the MSCs and/or the cells from a mesenchymal fraction are cultured in the presence of at least one agent which activates the Wnt canonical pathway.

5. Multipotent progenitor cells obtainable by the methods of any one of claims 3-4, wherein the progenitor cells express PDGFR-alpha and/or HLA-G.

6. A method for differentiating multipotent progenitor cells, optionally multipotent progenitor cells according to claim 5, into cardiac cells, comprising the step of culturing multipotent progenitor cells in the presence of at least one laminin selected from the group comprising laminin 111, laminin 121, laminin 211, laminin 221, and any combination thereof.

7. Cardiac cells obtainable by the method of claim 6, characterized in that:
(i) the cardiac cells express α-actinin, Troponin T, and Troponin I; and,
(ii) the cardiac cells are spontaneously beating at 40-100 beats per minute.

8. Cardiac progenitor cells, optionally obtainable by the method of claims 3-4, wherein the cardiac progenitor cells are characterized in that they are positive for at least one of Isl1, Sox2, SSEA1, Nanog, Tra-1, Brachyury T, CXCR4, PDGFRα, and HLA-G.

9. Cardiac progenitor cells according to claim 8, wherein the cells express CXCR4 and arrange in an elongated fashion in ischemic heart tissue.

10. MSCs, cells obtainable from a mesenchymal fraction, progenitor cells, cardiac progenitor cells or cardiac cells, characterized in that said cells are coated by at least one exogenously derived laminin selected from the group comprising LN-511, LN-521, LN-421, LN-411, LN-111, LN-121, LN-221, LN-211

11. A composition and/or a kit comprising:
(i) at least one laminin comprising an α5 chain; and,
(ii) at least one laminin comprising an α4 chain.

12. The composition and/or the kit according to claim 11, further comprising at least one agent which activates the Wnt canonical pathway.

13. The composition and/or the kit according to any one of claims 11-12, further comprising at least one agent which activates the Wnt canonical pathway.

14. The composition and/or the kit according to any one of claims 11-13, further comprising at least one laminin selected from the group comprising laminin 111, laminin 211, laminin 221, and any combination thereof, said at least one laminin preferably comprised in a separate container or on a separate surface suitable for cell culture.

15. Cells according to any one of claims 5 and 7-10 for use in medicine.

16. The cells according to any one of claims 5 and 7-10, for use in the treatment and/or prophylaxis of heart insufficiency, heart failure, myocardial infarction, congenital heart disease, myocarditis, valve dysfunction, acute respiratory distress syndrome (ARDS), graft-vs.-host disease (GvHD), solid organ rejections and/or rejections of cell and/or tissue transplants, inflammatory bowel diseases such as Crohn's disease and ulcerative colitis, rheumatoid diseases such as arthritis, any type of inflammation-driven or immunologically induced disease such as multiple sclerosis, ALS, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, or essentially any type of organ failure such as kidney failure, liver failure, lung failure, heart failure, and/or any combination thereof.

17. A pharmaceutical composition comprising the cells according to any one of claims 5 and 7-10 and at least one pharmaceutically acceptable excipient.

18. The pharmaceutical composition according to claim 17, further comprising at least one laminin.
